(19) United States
(12) Patent Application Publication
(10) Pub. No.: US 2023/0405248 A1
(43) Pub. Date: Dec. 21, 2023

(54) Respiratory Tidal Volume Monitor and Feedback Device

(71) Applicant: Board of Supervisors of Louisiana State University and Agricultural and Mechanical College, Baton Rouge, LA (US)

(72) Inventors: Johnathan Steven Alexander, Shreveport, LA (US); Luke A. White, Shreveport, LA (US); Giovanni Francesco Solitro, Shreveport, LA (US); Benjamin Maxey, Shreveport, LA (US); Steven A. Conrad, Shreveport, LA (US)

(73) Assignee: Board of Supervisors of Louisiana State University and Agricultural and Mechanical College, Baton Rouge, LA (US)

(21) Appl. No.: 18/241,548

(22) Filed: Sep. 1, 2023

Related U.S. Application Data
(63) Continuation of application No. PCT/US2022/027718, filed on May 4, 2022.
(60) Provisional application No. 63/156,773, filed on Mar. 4, 2021.

(51) Int. Cl. A61M 16/00; A61M 16/08; A61M 16/06; G09B 23/28

(52) U.S. Cl. CPC A61M 16/0051; A61M 16/0084; A61M 16/0816; A61M 16/06; G09B 23/28; A61M 2016/0033; A61M 2205/581; A61M 2205/584; A61M 2205/502; A61M 2205/15; A61M 2230/40; A61M 2205/3379

(57) ABSTRACT

A respiratory tidal volume monitor and feedback device (RTVMFD) comprising a frame, a flow channel tube attached to the frame, a mass flow sensor disposed on the flow channel tube to detect air flow through the flow channel tube, a microcontroller unit attached to the frame and electrically connected to the mass flow sensor via a bus, the microcontroller unit having a system processor, a system memory, and a system clock, a visual display attached to a top of the frame, the visual display being electrically connected to the bus. According to a further embodiment, the RTVMFD further comprises an audio output attached to the frame and electrically connected to the bus.

### CROSS REFERENCE TO RELATED APPLICATIONS/PRIORITY

The present invention claims priority to U.S. Provisional Patent Application No. 63/156,773 filed Mar. 4, 2021, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

Bag valve mask (BVM) ventilation remains one of the most widely used methods for providing short-term and emergency ventilation. BVMs like the AMBU® Bag are cost-effective and easily transported but are also highly susceptible to user error, especially in high-stress emergency situations. Inaccurate and inappropriate ventilation has the potential to greatly harm patients through hyper- and hypoventilation. Healthcare providers deliver emergency breaths with a BVM to a patient by hand-squeezing an inflated bag connected to a mask that fits over a patient's nose and mouth. Although simple in concept, effective ventilation with BVM systems requires extensive training, and potential misuse can significantly diminish the BVM's efficacy and safety. Hypoventilation and inadequate gas exchange are valid concerns during manual ventilation, but hyperventilation remains a more common user error and can also lead to injury. Delivering too great a tidal volume (the volume of air provided to the patient during one round of inspiration) increases the risk for potentially serious adverse effects including hemodynamic changes, lung injury, acute barotrauma, and direct volutrauma to the lungs leading to pneumothorax, gastric insufflation and aspiration. Though BVMs are used widely to resuscitate and ventilate critically ill patients, they are causing actual harm in everyday use. Healthcare providers at all levels are generally very ineffective at providing appropriate ventilations with bag valve masks, normally providing tidal volumes that significantly exceed safe thresholds for lung protective ventilation.

### SUMMARY

Wherefore, it is an object of embodiments the present invention to overcome one or more of the above mentioned shortcomings and drawbacks associated with the current technology. The present invention is directed to a respiratory tidal volume monitor and feedback devices (RTVMFD) and methods of use comprising a frame, a flow channel tube attached to the frame, a mass flow sensor disposed on the flow channel tube to detect air flow through the flow channel tube, a microcontroller unit attached to the frame and electrically connected to the mass flow sensor via a bus, the microcontroller unit having a system processor, a system memory, and a system clock, a visual display attached to a top of the frame, the visual display being electrically connected to the bus. According to a further embodiment, the RTVMFD further comprises an audio output attached to the frame and electrically connected to the bus. According to a further embodiment, the visual display is a plurality of LED pixels. According to a further embodiment, the LED pixels are arranged in a ring. According to a further embodiment, the RTVMFD further comprises a plurality of target volume (V_{T}) indicia disposed on a ring guard, with each of the V_{T} indicia being adjacent to a respective LED pixel representing a respective target volume for the each of the V_{T} indicia. According to a further embodiment, the system processor is configured to execute instructions to receive mass flow data from the mass flow sensor during an inspiration, cause the LED pixels to illuminate sequentially and in a proportional number to a tidal volume of inspirated air represented by the mass flow data. According to a further embodiment, system processor is further configured to execute instructions to cause the LED pixels to change colors of illumination in response to the tidal volume of inspirated air. According to a further embodiment, the complete LED pixel ring represents 100% of a target tidal volume, and the LED pixels sequentially and proportionally illuminate forming an increasing arc length of illuminated LED pixels around a circumference of the ring as the measured tidal volume of inspirated air for each inspiration ranges from 0.0 mL to the target tidal volume. According to a further embodiment, the LED pixels change from a first color of illumination to a second color of illumination when the measured tidal volume reaches the target tidal volume. According to a further embodiment, the RTVMFD further comprises the system processor determining if the RTVMFD is in an expiration state, an idle state, or an inspiration state. According to a further embodiment, the RTVMFD further comprises when the RTVMFD is in an expiration state, the system processor checks for leaks by receiving expiration mass flow data from the mass flow sensor during a current expiration state, computing an expiratory tidal volume from the expiration mass flow data, and if the expiratory tidal volume is between 60.0% and 0.0% of an immediately previous inspiratory volume, determine that a leak is present. According to a further embodiment, the RTVMFD further comprises the system processor causing one of an audible cue, a visual cue, and both an audible cue and a visual cue to be generated when a leak is determined to be present. According to a further embodiment, the RTVMFD further comprises the system processor determining a flow rate of air during inspiration, and when the flow rate is faster than an upper limit, determine that the inspiration rate is too fast, and when the flow rate is slower than a lower limit, determine that the inspiration rate is too slow. According to a further embodiment, the system processor causes the speakers to generate a verbal audio cue to alert the user to bag faster if the inspiration rate is determined to be too slow, and generate a verbal audio cue to alert the user to bag slower if the inspiration rate is determined to be too fast.

The presently disclosed invention is further related to a method of training a user to ventilate a patient comprising providing the user with a respiratory tidal volume monitor and feedback device (RTVMFD) and a Bag-Valve-Mask (BVM) functionally connected to one another, wherein the RTVMFD has a frame, a flow channel tube attached to the frame, a mass flow sensor disposed on the flow channel tube to detect air flow through the flow channel tube, a microcontroller unit attached to the frame and electrically connected to the mass flow sensor via a bus, and a visual display attached to a top of the frame, with the visual display being electrically connected to the bus, selecting a target tidal volume, operating the BMV attempting to deliver the target tidal volume through the RTVMFD, providing the user with substantially real time tidal volume feedback on success in reaching the target tidal volume with each inspiration delivered. According to a further embodiment, the tidal volume feedback is in the form of an illumination of a progressive number of LED pixels arranged in a ring shape on a surface of the frame, where none of the LED pixels illuminated represents delivering a tidal volume of 0.0 mL and all of the LED pixels illuminated represents delivering a tidal volume equal to the target tidal volume. According to a further embodiment, the tidal volume feedback is in the form of an illumination color change of the LED pixels when the target tidal volume is reached. According to a further embodiment, the method further comprises the system processor tracking a duration of inspiration with each inspiration delivered, determining if the duration of inspiration is less than, within, or greater than a target duration of inspiration window, causing one of the visual display or a speaker to provide the user with substantially real time duration of inspiration feedback when the duration of inspiration is outside of the duration of inspiration window. According to a further embodiment, the duration of inspiration feedback is in the form of a verbal cue that the duration of inspiration is short when the duration of inspiration less that the duration of inspiration window, and in the form of a verbal cue that the duration of inspiration is too long when the duration of inspiration is greater the inspiration window.

The presently disclosed invention further relates to a respiratory tidal volume monitor and feedback devices (RTVMFD) and methods of use comprising a frame, a flow channel tube attached to the frame, with a first end of the flow channel tube accessible for functional attachment to Bag-Valve-Mask (BVM) system bag outlet, a mask connector, with a first end of the mask connector one of attached to and of unitary construction with a second end of the flow channel tube, and a second end of the mask connector accessible for functional connection to a BVM system mask, a mass flow sensor disposed on the flow channel tube to detect air flow through the flow channel tube, a microcontroller unit attached to the frame and electrically connected to the mass flow sensor via a bus, the microcontroller unit having a system processor, a system memory, and a system clock, an audio processor electronically connected to the bus, a visual display attached to a top of the frame, the visual display being electrically connected to the bus, wherein the visual display is plurality of LED pixels arranged in a ring shape, a speaker attached to the frame and electrically connected to the bus, a plurality of target volume (V_{T}) indicia disposed on a ring guard, with each of the V_{T} indicia being adjacent to a respective LED pixel representing a respective target volume for the each of the V_{T} indicia, the system processor being configured to execute instructions to receive mass flow data from the mass flow sensor during an inspiration, cause the LED pixels to illuminate sequentially and in a proportional number to a tidal volume of inspirated air represented by the mass flow data, wherein the complete LED pixel ring represents 100% of a target tidal volume, and the LED pixels sequentially and proportionally illuminate forming an increasing arc length of illuminated LED pixels around a circumference of the ring as the measured tidal volume of inspirated air for each inspiration ranges from 0.0 mL to the target tidal volume, cause the LED pixels to change colors of illumination from a first color to a second color in response to tidal volume of inspirated air increasing from below 50.0% of target tidal volume to more than 50.0% of target tidal volume, cause the LED pixels to change colors of illumination from the second color to a third color in response to tidal volume of inspirated air increasing from below 100.0% of target tidal volume to more than 100.0% of target tidal volume, determine if the RTVMFD is in an expiration state, an idle state, or an inspiration state, and when the RTVMFD is in an expiration state, the system processor checking for leaks by receiving mass flow data from the mass flow sensor during an expiration, computing an expiratory tidal volume from the expiration mass flow data, and when the expiratory tidal volume is less than 50.0% of an immediately previous inspiratory volume, determine that a leak is present, and causing a verbal audible cue to be generated when a leak is determined to be present; track a duration of inspiration with each inspiration delivered, determine if the duration of inspiration is less than, within, or greater than a target duration of inspiration window, cause one of the visual display or speaker to provide the user with a substantially real time duration of inspiration feedback when the duration of inspiration is outside of the duration of inspiration window, wherein the duration of inspiration feedback is in the form of a verbal cue that the duration of inspiration is short when the duration of inspiration less that the duration of inspiration window, and in the form of a verbal cue that the duration of inspiration is too long when the duration of inspiration is greater the respiration rate window, and cause the audio processor to cause the speaker to generate a verbal audio cue instructing the user bag initiate bagging at regular timed intervals.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that while the accompanying drawings are to scale for one or more embodiments of the disclosed invention, the emphasis is rater placed on illustrating the principles of the invention. The invention will now be described, by way of example, with reference to the accompanying drawings in which:

FIG. 1 is an isometric view of an embodiment of the respiratory tidal volume monitor and feedback device as presently disclosed incorporated into a bag valve mask system.

FIG. 2 is an isometric view of the respiratory tidal volume monitor and feedback device of FIG. 1.

FIG. 3 is an isometric view of the respiratory tidal volume monitor and feedback device of FIG. 1 without the housing cover.

FIG. 4 is an exploded isometric view of the respiratory tidal volume monitor and feedback device of FIG. 1.

FIG. 5A is a circuit diagram for the respiratory tidal volume monitor and feedback device. Components include Arduino Nano Every (ABX00028), Sensirion® digital flow sensor (SPD31), 5V boost module (TPS61023), LED ring (24xWS2812), MP3 module (DFR0299), and miniature speaker. The 5V booster/regulator powers the Arduino, LED ring, and flow sensor, while the MP3 module and speaker receive power directly from the batteries.

FIG. 5B is a software flowchart diagram for the respiratory tidal volume monitor and feedback device. The main software loop consists of three phases indicated by the areas separated by the two horizontal dashed lines: (Phase 1) updating timers, playing appropriate audio cues, and reading data from the flow meter; (Phase 2) updating the device's state and performing the corresponding calculations; and (Phase 3) updating displays, either current tidal volume or status displays. Exp.=expiration, insp.=inspiration, V_{T}=tidal volume.

FIG. 6 is a schematic representation of a state diagram for the respiratory tidal volume monitor and feedback device. The respiratory tidal volume monitor and feedback device operates in three states: idle, inspiration, and expiration. Changes in state are determined largely by the direction of air flow through the device. Of the previous 25 flow measurements, the ‘positive flow count’ represents the number of measurements that were greater than 0 (directed into the patient), and the ‘negative flow count’ represents the number that were less than 0 (directed out of the patient). V_{T} represents the current delivered tidal volume, in mL.

FIG. 7 is a photograph of an assembled respiratory tidal volume monitor and feedback device prototype.

FIG. 8 is a photograph of a demonstration of the respiratory tidal volume monitor and feedback device with a bag valve mask (BVM) and manikin.

FIGS. 9A-9F are photographs of the respiratory tidal volume monitor and feedback device LED ring producing signals during tidal volume delivery. FIGS. 9A-9E show visual cues produced by the LED ring at varying levels of target V_{T} achieved (shown as % V_{T}) of 0% (FIG. 9A), 25% (FIG. 9B), 50% (FIG. 9C), 75% (FIG. 9D), and 100% (FIG. 9E). As an inspiration is delivered, the LEDs light up consecutively around the ring, changing from green (between 0% to 50% target V_{T}), to yellow (50% to 100%), and finally to red (>100%) signaling that the full target tidal volume has been delivered. V_{T}=tidal volume, Vₑₓₚ=expiratory volume. FIG. 9F shows LED display during status mode. In this mode, the top half of the LED ring displays the current tidal volume setting via purple lights aligned with volumes (in mL) printed into the casing of the respiratory tidal volume monitor and feedback device. The bottom half of the LED ring displays battery life, with green, yellow, and red together representing full battery, yellow and red together representing medium battery life, and red alone representing low battery.

FIG. 10 is a graphic representation of the respiratory tidal volume monitor and feedback device visual and verbal audio cues (in quotations), timings, and triggers during the 6 second respiratory cycle. Time in inspiration and expiration represented with green and red rectangles with calculations performed in real-time (text inside boxes).

FIGS. 11A and 11B are two charts showing that the respiratory tidal volume monitor and feedback device accurately measures airflow rate. FIG. 11A shows average measured flow from the respiratory tidal volume monitor and feedback device versus the flow sensor connected to the computer at different constant flow rates sampled over 100 ms. Solid line indicates best-fit linear regression (R²>0.99); dotted line represents exact match between measurements. FIG. 11B shows Bland-Altman plot of measured flows between the respiratory tidal volume monitor and feedback device and computer (dotted line represents 95% limits of agreement; percent Difference calculated as % Difference=(respiratory tidal volume monitor and feedback device flow−computer flow)/average flow).

FIGS. 12A-12 C show flow sampling rate with the respiratory tidal volume monitor and feedback device was sufficient for accurate tidal volume measurement. FIG. 12A shows flow sampling rate of the respiratory tidal volume monitor and feedback device measured during multiple (n=43) 1-second intervals. Error bars indicate mean±standard deviation. FIG. 12B show flow measurement profile captured by the computer sensor (black line) of a single inspiratory cycle from a BVM delivered to a test lung (measured tᵢₙₛₚ=1.08 s and V_{T}=506 mL), and simulated flow measurement profiles at captured at lower sampling rates (shown are sampling rates at 3, 10, 100, and 1000 samples of the full inspiratory time). FIG. 12C shows Log-Log plot of difference in calculated V_{T} from the simulated measurement profiles (3 to 10 samples at 1-sample intervals, 10 to 100 samples at 10 sample intervals, and 100 to 2100 samples at 100 sample intervals) to V_{T} calculated from the computer sensor versus the sampling rate (solid line indicates best-fit linear regression, R²=0.92). Red point indicates the predicted error between the respiratory tidal volume monitor and feedback device V_{T} and computer, given the measured mean sample rate for the respiratory tidal volume monitor and feedback device (716 Hz) and assuming flow measurements were in exact agreement between the respiratory tidal volume monitor and feedback device and computer sensor for all time points.

FIGS. 13A-13C are charts showing the respiratory tidal volume monitor and feedback device accurately measures different tidal volumes delivered with a mechanical ventilator at varying inspiratory times. FIG. 13A shows average measured tidal volumes (V_{T}) by the respiratory tidal volume monitor and feedback device versus the computer flow sensor for varying inspiratory times (tᵢₙₛₚ=0.5, 1.0, and 2.0 s) and tidal volumes (for tᵢₙₛₚ=1.0 and 2.0 s, V_{T}=300-900 mL in 50 mL increments; for tᵢₙₛₚ=0.5 s, V_{T}=300-750 mL in 50 mL increments) delivered by the Carestation® mechanical ventilator set in volume control ventilation (respiratory rate=10 breaths/min, positive end expiratory pressure=0 cm H₂O) connected to a test lung for 10 consecutive respiratory cycles (dotted line indicates perfect match). FIG. 13B shows Bland-Altman plots stratified by inspiratory time (dotted lines indicate the 95% limits of agreement; percent difference calculated as % Difference=(respiratory tidal volume monitor and feedback device flow−computer flow)/average flow). FIG. 13C shows tidal volume difference versus the average flow measured during each inspiratory cycle (solid line indicates best-fit linear regression, R²=0.98).

FIG. 14 is a table showing verbal audio cue upper and lower bound triggering. Superscripts are as follows: a=V_{T} tidal volumes, measured in mL, at which each audio cue was tested (300, 500, and 750 mL). b=upper bounds (UB) and lower bounds (LB) for inspiratory times (in seconds) that trigger the “Bag slower” and “Bag faster” audio cues for a given tidal volume. c=Upper (UB) and lower bounds (LB) for leaks (in %) that trigger the “Leak detected” audio cue for a given tidal volume. d=The trigger for the “Bag slower” audio cue was not tested at 750 mL because the maximum pressure allowed by that embodiment's mechanical ventilator was exceeded. NA not available.

FIGS. 15A-15D are eight charts showing that accurate tidal volume delivery was improved with respiratory tidal volume monitor and feedback device use, with volumes of 500 mL (FIG. 15A), 750 mL (FIG. 15B), 300 mL (FIG. 15C), and 20 mL (FIG. 15D), and with each volume showing a chart of tidal volume delivered by distinct users (top charts) and change in tidal volume delivered by the same set of users (bottom charts) each using a bag valve mask (BVM) system without (Control) and with the respiratory tidal volume monitor and feedback device.

FIGS. 16A-16D are eight charts showing that respiratory rate was improved for users with the respiratory tidal volume monitor and feedback device use, with volumes of 500 mL (FIG. 16A), 750 mL (FIG. 16B), 300 mL (FIG. 16C), and 20 mL (FIG. 16D), and with each volume showing a chart of respiratory rate for distinct users (top charts) and change in respiratory rate for the same users (bottom charts) each using a bag valve mask (BVM) system without (Control) and with the respiratory tidal volume monitor and feedback device.

FIG. 17 is a table showing that mean inter-user variability was reduced with respiratory tidal volume monitor and feedback device (RTVMFD) use.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

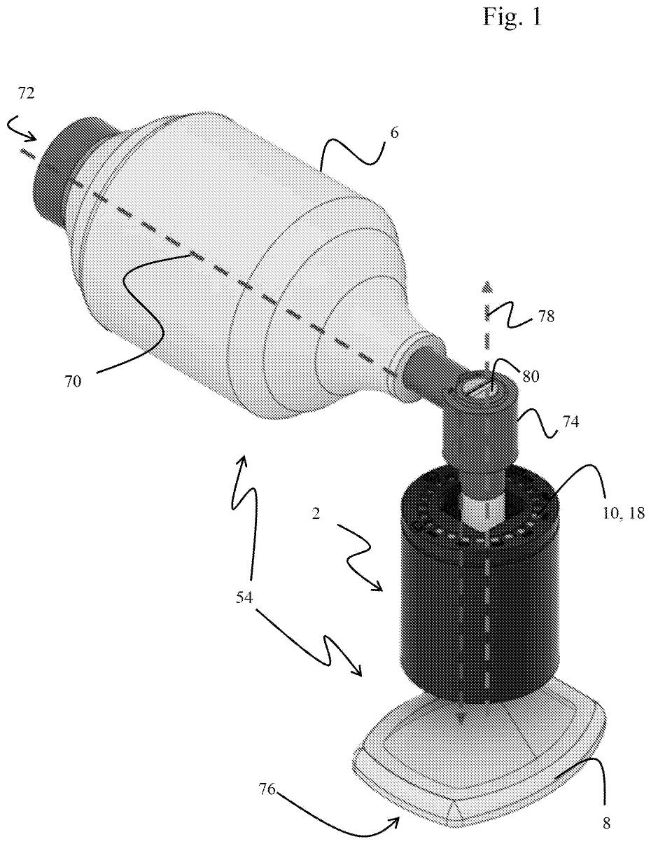

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. Where spatial directions are given, for example above, below, top, and bottom, such directions refer to the respiratory tidal volume monitor and feedback device as represented in FIG. 1, unless identified otherwise.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “substantially” means that the property is within 80% of its desired value. In other embodiments, “substantially” means that the property is within 90% of its desired value. In other embodiments, “substantially” means that the property is within 95% of its desired value. In other embodiments, “substantially” means that the property is within 99% of its desired value. For example, the term “substantially complete” means that a process is at least 80% complete, for example. In other embodiments, the term “substantially complete” means that a process is at least 90% complete, for example. In other embodiments, the term “substantially complete” means that a process is at least 95% complete, for example. In other embodiments, the term “substantially complete” means that a process is at least 99% complete, for example.

The term “substantially” includes a value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

The term “about” includes when value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

In addition, the invention does not require that all the advantageous features and all the advantages of any of the embodiments need to be incorporated into every embodiment of the invention.

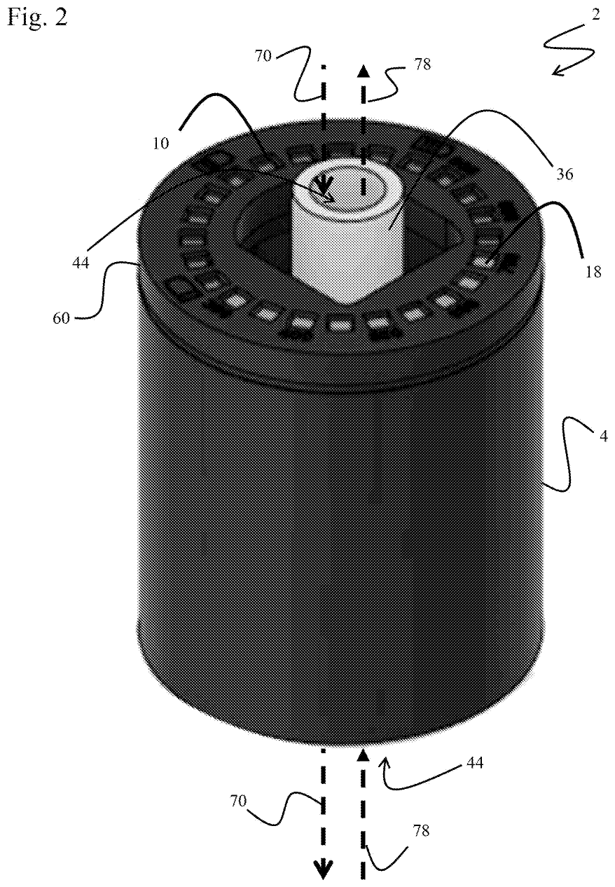

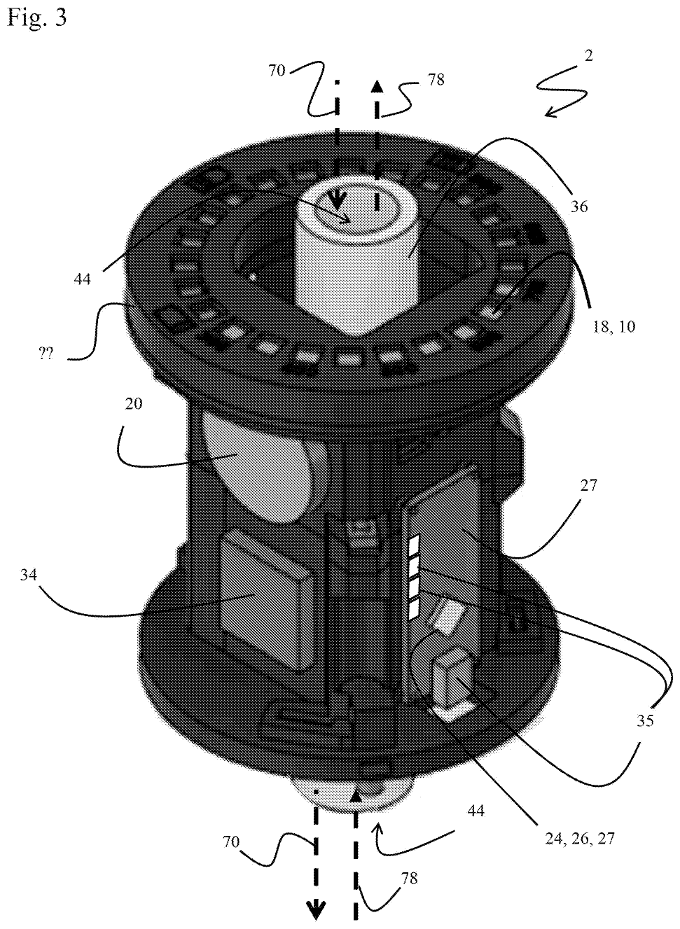

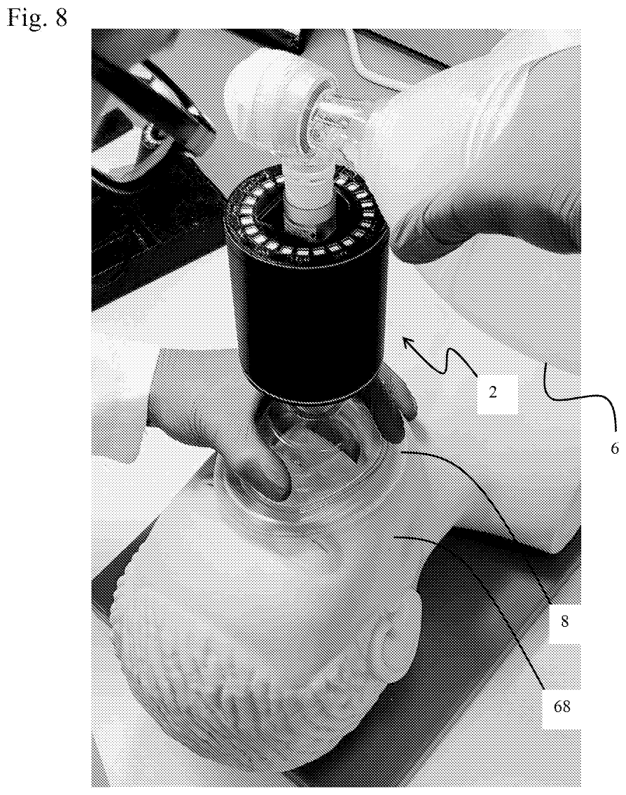

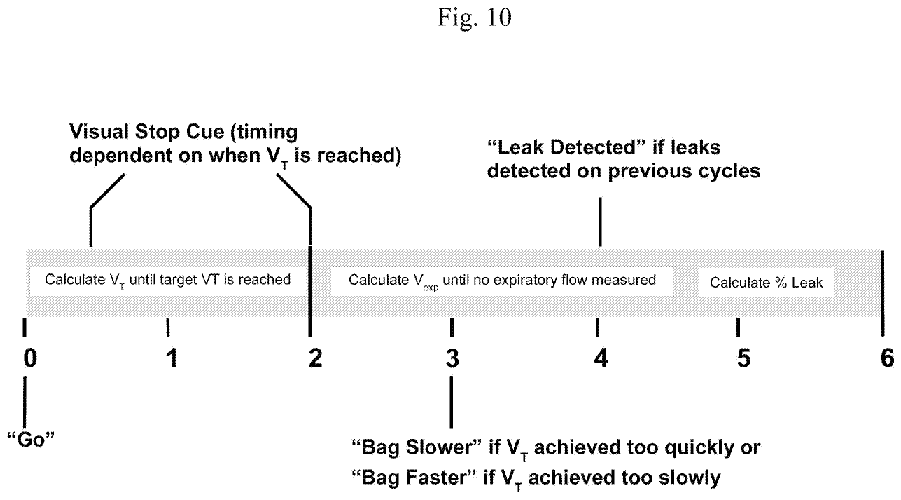

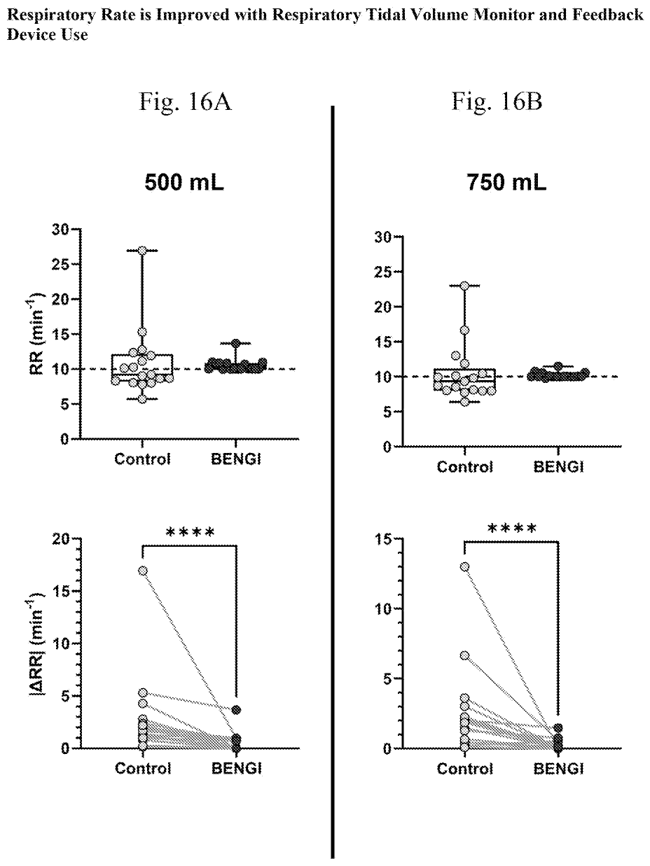

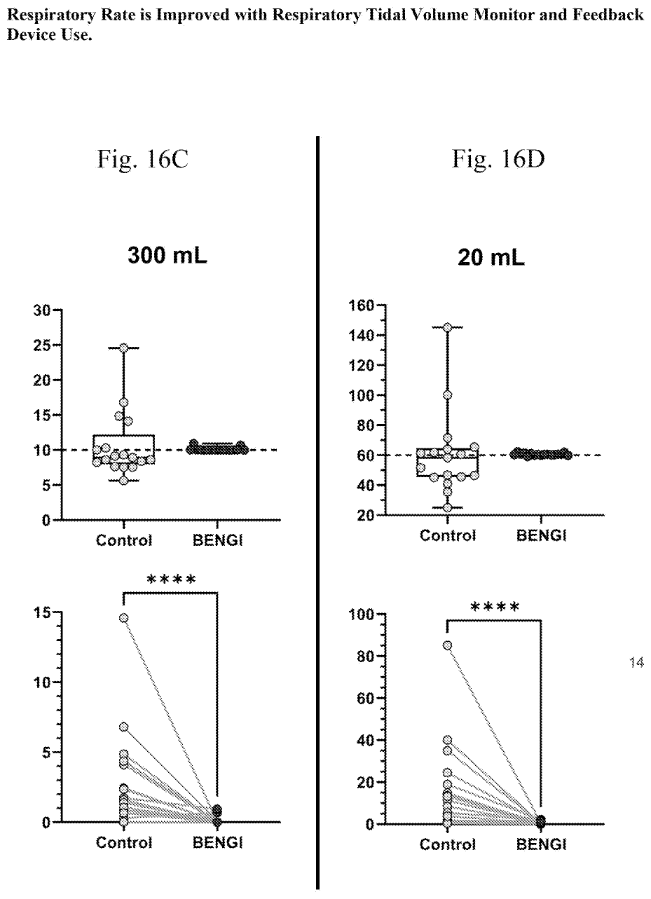

Turning now to FIGS. 1-17, a brief description concerning the various components of the present invention will now be briefly discussed.

When a healthcare provider ventilates a patient with a BVM, the provider places a mask over the patient's mouth and manually squeezes a bag to “breath” for the patient. When the bag is squeezed, air passes through a mask and into the patient's airway, allowing for oxygenation and carbon dioxide removal. Release of pressure on the bag allows air to flow through an outlet valve and exit the patient's lungs. Indications for manual ventilation include both in-hospital and out-of-hospital cardiopulmonary resuscitation, as well as short-term ventilation solutions (such as during a tracheal intubation procedure prior to mechanical ventilator connection or transporting patients around the hospital or to another facility) and neonatal respiratory support after delivery. Of particular relevance is the BVM's potential utility in austere medical environments where access to advanced respiratory support is limited by medical equipment (mechanical ventilators, capnographs, and pulse oximeters), supplies (breathing circuits, personal protective equipment, disinfectants) and infrastructure (electrical power and pressurized oxygen/air), exacerbated by supply chain disruption during the ongoing COVID-19 pandemic.

Even ‘properly’ trained personnel tend to ‘over-bag’ patients, meaning that tidal volumes are too high, and/or the ventilation rate is too fast. For example, studies estimate that in up to 80% of cases healthcare providers (regardless of level of training) will over-ventilate patients. The International Liaison Committee on Resuscitation (ILCOR) recommends ventilating at a tidal volume of 400 to 600 mL, at a rate of 8 to min⁻¹, and data show that lower volume ventilation can yield lung-protective effects in several clinical scenarios. In one manikin study, 20 ambulance personnel bagged an adult manikin at a mean tidal volume of 746±221 mL (significantly higher than the study's defined target tidal volume upper bound of 600 mL) with a respiratory rate of ±4.5 breaths/minute. In another manikin study with 140 participants ranging from first-aid workers to physicians, excessive ventilation (defined by tidal volume greater than 600 mL and respiratory rate above 15 breaths/min) occurred in over 79% of tests. For manual ventilation of pediatric patients, one study simulating 72 inpatient pediatric CPR events demonstrated that providers on average bagged at double the study's target ventilation rate of 20 breaths per minute. A similar manikin study in neonates showed that average delivered tidal volumes were over 150% of the European Resuscitation Council's recommended upper bound. Clinical studies examining real-life resuscitation events also consistently show the tendency for providers to over-bag patients.

The inventors concluded that without real time objective feedback, providers cannot always appreciate the quality of their respirations during manual ventilation. The inventors disclose herein a user-friendly, cost-effective tidal volume monitoring feedback device 2 design optimized for high-volume, resource-scarce emergent care—a healthcare setting that has become increasingly common amid the COVID-19 pandemic. Embodiments of the disclosed respiratory tidal volume monitor and feedback device 2, a BVM emergency narration-guided instrument, communicates to the user through visual and spoken audible cues to alert the user when a target tidal volume has been reached, when to begin and end inspirations, when peak airflow is too high or low, and if a mask leak is detected. The assembly and components of multiple exemplary embodiments of respiratory tidal volume monitor and feedback device 2 are herein disclosed, including different algorithms utilized by the device, and tests of the accuracy of the implemented algorithms through a variety of preclinical benchtop tests. Further, methods of training healthcare personnel in delivering more accurate tidal volume and respiratory rates on BVMs through use of the respiratory tidal volume monitor and feedback device 2 is disclosed.

Rapid design prototyping and revision for the respiratory tidal volume monitor and feedback device housing 4 was achieved with in-house fused filament deposition 3D printing. The inventors designed the respiratory tidal volume monitor and feedback device 2 to be radially symmetric so that attachment to the bag 6 and mask 8 could be achieved rapidly without need of adjustment to effectively view visual cues displayed by a visual display 10. A small digital mass flow sensor 12 measures airflow delivered during an inspiration, and the respiratory tidal volume monitor and feedback device calculates in real-time the total tidal volume (V_{T}) achieved during a delivered breath. In one embodiment of the respiratory tidal volume monitor and feedback device, the user can quickly and easily set a range of target V_{T} between 300 and 900 mL in 50 mL increments via a push button settings button 14 on, for example a bottom surface 16 of the device 2. In the embodiment shown, the visual display 10 is an LED ring 18 that informs the user of the cumulative V_{T} delivered during each breath, and an audio output 20, in the form of a speaker in the embodiment shown, plays audio cues, preferably verbal audio cues, that alert the user when to begin each respiration. Additional audio cues give feedback on the quality of the user's respirations and instruct the user to bag more quickly or slowly if inspiratory times are too long or short, respectively. A verbal audio cue is understood to include a nuncupative word or group of words, such as a linguistic form, generated by the audio output 20, that preferably informs the user in the user's language as to an action to take and/or to information about the state of the patient, the state of the device 2, and/or the operation of the device 2. Audio cues, including verbal audio cues may also be accompanied by textual displays of the words in the audio cue by the visual display 10.

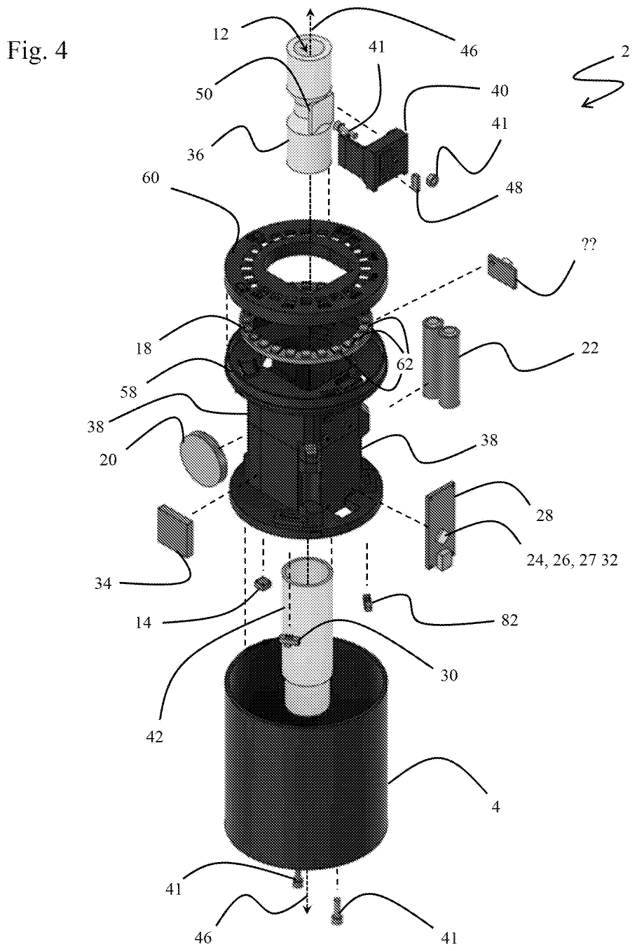

The power source 22 for the unit 2 is preferably 3 AAA batteries (even though only two batteries are shown in FIG. 4), and a battery health check feature has been implemented. Other power sources are envisioned, such as different size, voltage, amperage, and or number of batteries, for example.

Control System. In the embodiment shown, a system processor 24, a system memory 26, and a system clock 27 are preferably provided as part of a microcontroller unit 28, such as an Arduino Nano Every (Arduino®; ABX00028), as shown. The Arduino Nano Every uses an ATMega4809 microcontroller with 48 kB of CPU flash memory, 6 kB of SRAM, 256-byte EEPROM, and a 20 MHz clock. Ease of implementation and abundant product support are major advantages of using Arduino boards. They are readily reprogrammable via serial connection and can report data over serial connection during testing, making them an ideal choice for rapid prototyping. The computation required for this application is relatively light, and the hardware specifications of the Arduino Nano Every are more than sufficient, though other computer processors, computer memories, and microcontrollers may be used. Inputs to the respiratory tidal volume monitor and feedback device 2 in the embodiment shown include a digital mass flow sensor 12, an audio control button 30, such as a single pole single throw switch to mute audio, a battery life circuit 32 that measures battery life—included in the Arduino Nano Every in the embodiment shown, and a settings button 14 switch to adjust user settings and cause the visual display 10 to display of battery life. Outputs include a speaker 20 preferably with an audio processor 34 such as a MP3 player, as well as a visual display 10 such as a LED pixel ring 18 to display tidal volume data or current settings and battery life. The microcontroller unit 28 communicates with the various inputs and outputs via one or more electronic connections 35.

Digital Mass Flow Sensor 12. A core functionality of the respiratory tidal volume monitor and feedback device 2 involves measuring a volume of air that passes through the device 2. According to one embodiment, the respiratory tidal volume monitor and feedback device 2 utilizes a mass flow sensor 12 (Sensirion®; SDP3x) housed in a preferably pre-made flow channel tube 36 (Sensirion®; SFM3300-D), preferably formed from plastic or metal, to make such measurements. In the embodiment shown, the mass flow sensor 12 is positioned on the interior of the flow channel tube 36 and is not visible. The flow channel tube 36 preferably attaches to a central frame 38 of the respiratory tidal volume monitor and feedback device 2 via a clip 40 that is secured to the central frame 38 by a fastener 41, such as a chemical fastener like a glue or a mechanical fastener like a nut, bolt, screw, washer, key and key-way, stud, rivet, anchor, nail, insert, retaining ring, clevis pin and cotter pin, for example. A substantially cylindrical mask connector 42 is attached to a bottom end of the flow channel tube 36. The mask connector 42 is preferably formed from plastic or metal. The flow channel tube 36 and the mask connector 42 together form a ventilation channel 44 and define a central axis 46. Preferably spring-loaded metal pins 48 are affixed inside the clip 40 make electrical contact with pads 50 on the surface of the flow channel tube 36. The pins 48 traverse a space in the wall of the ventilation channel 44 to allow physical and electrical connection to the rest of the respiratory tidal volume monitor and feedback device 2, and data output from the mass flow sensor 12 is transmitted over a serial communication bus 52, such as I2C, connecting the inputs devices, output devices, and power to the microcontroller unit 28.

The elements of the ventilation channel 44, including the flow channel tube 36 with mass flow sensor 12 and mask connector 42 are all preferably designed to be releasably attached to the respiratory tidal volume monitor and feedback device 2, so they may be replaced between uses so as to minimizes cross-contamination. When the respiratory tidal volume monitor and feedback device 2 is connected to a BVM system 54, preferably the only portion of the device in contact with the respiratory circuit is the ventilation channel 44. The flow channel tube 36 with mass flow sensor 12 and mask connector 42 are preferably disposable, allowing all parts that are exposed to patients to be completely removed and disposed or cleaned between patient use. Patient safety, as well as ease in transitioning the device from one patient to another, were some motivations of this design in this embodiment.

Audio processing and output: Audio cues are played through an audio processor 34 connected to an audio output 20. In the embodiment shown a miniature MP3 module (DFRobot®; DFR0299) functions as the audio processor 34 connected to a small 8 Ω 0.5W metal speaker (Adafruit®; 1890), which functions as the audio output 20. One or both of the audio processor 34 and audio output 20 may be integrated onto the microcontroller unit 28, and the system processor 24 may also process the audio signals, not requiring a separate system processor 24 and audio processor 34. In the embodiment shown, the MP3 module 34 reads .mp3 files stored on a non-volatile memory card, such as an SD card (not shown) and drives the small metal speaker 20. In further embodiments, the audio files may be stored in the system memory 26 or in a memory unit on the audio processor 34, for example. Commands are sent to the MP3 module 34 from the system processor 24 via the bus 52 serial connection. The MP3 module 34 supports a variety of control modes, volume levels, and playback features, all of which may be used in future embodiments, but the embodiment shown may be limited to the playback of five tracks without any special features.

Power. All electronic components in the shown embodiment of the respiratory tidal volume monitor and feedback device may be designed to operate on a 5V input, even though in the embodiment shown, the device 2 is powered by a power source 22 consisting of 3 AAA batteries (only 4.5V in series). Adequate voltage is generated through a voltage booster 56, a 5V booster (Texas Instruments®; TPS61023) in the embodiment shown, which takes inputs of 2V to 5V and outputs a steady 5.2V. The decision to use AAA batteries was motivated largely by cost and space requirements, as power sources at and above 5V are generally significantly more expensive and larger. In future embodiments, the voltage booster 56 may be omitted by, for example, using other electronic components with a smaller voltage and or amperage draw. Initially, 2 AAA batteries 22 were used to save space, as 2 AAA batteries 22 were

sufficient to power the entire device 2 while muted. During testing, however, the inventors discovered that both the DFRobot MP3 module 34 and the LED ring 18 have substantial active current draw, and that the current output of the 5V booster 56 with 2 AAA batteries 22 was not sufficient to support both components 34, 18. Maximum current output from the 5V booster 56 is dependent on its supply voltage. At 3V input, the maximum output current is only ˜800 mA. Connecting a third AAA battery 22 in series almost doubles the value, raising the maximum current output of the 5V booster to 1400 mA, a value sufficient to power both components 34, 18 simultaneously. Connecting the MP3 module 34 directly to the batteries' 4.5V output further reduced the current output requirement from the 5V booster module. The respiratory tidal volume monitor and feedback device's 2 battery life check

feature measures the voltage of the 3 AAA batteries 22 directly using one of the Arduino's analog inputs 35. These inputs reference the board's supply voltage, and the booster's 56 output voltage is tightly regulated. Small fluctuations in the booster's 56 output voltage are not great enough to impact the battery check feature.

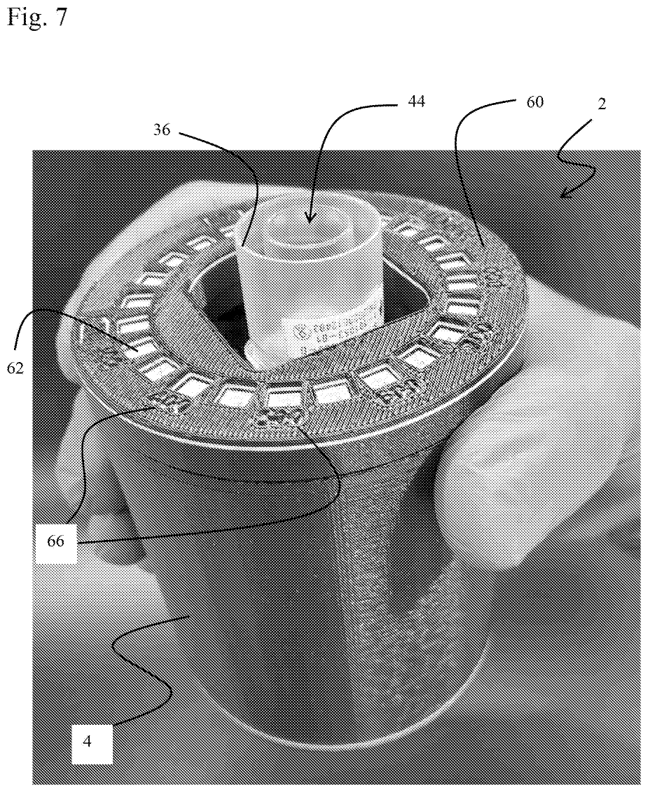

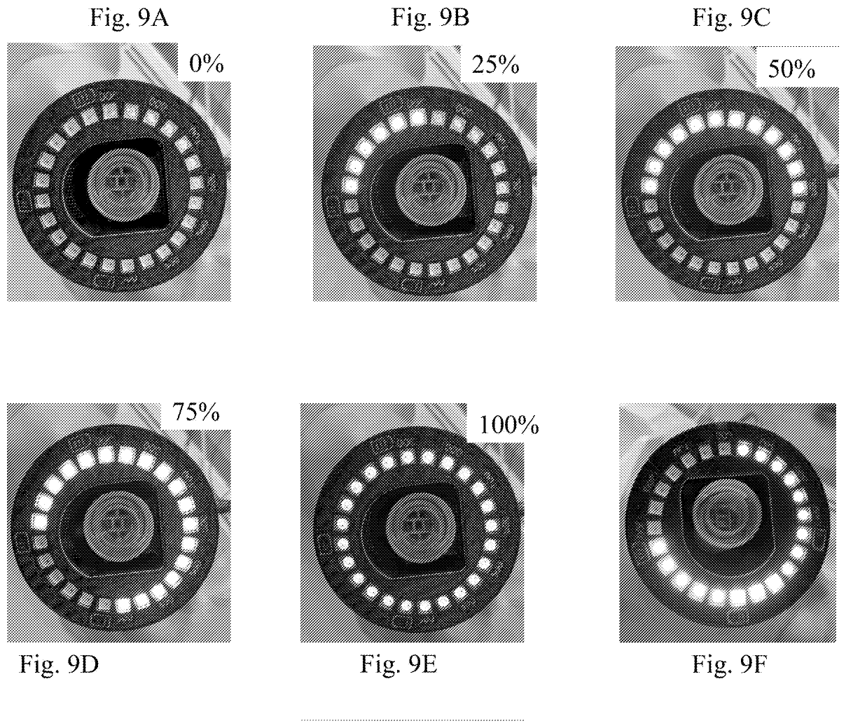

Visual Display: Visual cues are given to the user by the visual display 10. In the embodiment shown, the visual display is a NeoPixel® LED ring 18 (Adafruit®; 24xWS2812), which is affixed to the top surface 58 of the frame 38, and covered by a ring guard 60 that holds the LED ring 18 in place and protects the LED ring 18. The LED ring 18 consists of 24 LEDs or pixels 62, each with individually controlled brightness and RGB values. While display screens may be used in place of or in addition to the LED ring 18 for the visual display 10 in some embodiments, the use of LEDs versus a display screen for visual cues is simple, interpretable, robust, and easily visible in a variety of conditions while simultaneously keeping unit costs lower compared to the use of display screens, and allows for a longer battery life. The flexibility and specificity of control of this LED ring 18 allows for complex patterns when conveying data to the user. In normal operating mode, all pixels 62 are off by default. When the user begins delivering a breath through the respiratory tidal volume monitor and feedback device 2, pixels 62 begin lighting up, progressing clockwise around the LED ring 18. In one embodiment, the number and color of lit pixels 62 light up at a given time corresponds to the fraction of the target V_{T} that the user has delivered (See FIGS. 9A-9E). Upon pushing the settings button 14 located on a bottom surface 64 of the frame 38, the respiratory tidal volume monitor and feedback device 2 enters status mode. In status mode, a first portion of the pixels 62 (5 green, 3 yellow, and 3 red pixels 62 of bottom half of LED ring 18 of FIG. 9F) display a meter representing the current battery life or remaining charge of the device 2, while a second portion of the pixels 62 (7 unlight and 6 purple pixels 62 of top half of LED ring 18 in FIG. 9F) display the current V_{T} setting (See FIG. 9F). Preferably, permanent visual and/or tactile V_{T} indicia 66 are provided. In the embodiment shown, the V_{T} indicia 66 used are raised numbers 66 on the top surface of the ring guard, where the respective raised numbers are 66 radially aligned with pixels 62 in the LED ring 18 to indicate V_{T} settings, and subsequent presses of the settings button 14 cause the system processor 24 to cycle through the V_{T} settings, from 300 to 900 mL in 50 mL increments, for example, with pixels 62 adjacent to the V_{T} indicia 66 corresponding to the chosen setting lighting up with each selection. In FIG. 7 the V_{T} indicia 66 of raised numbers for 400 mL and 500 mL are indicated.

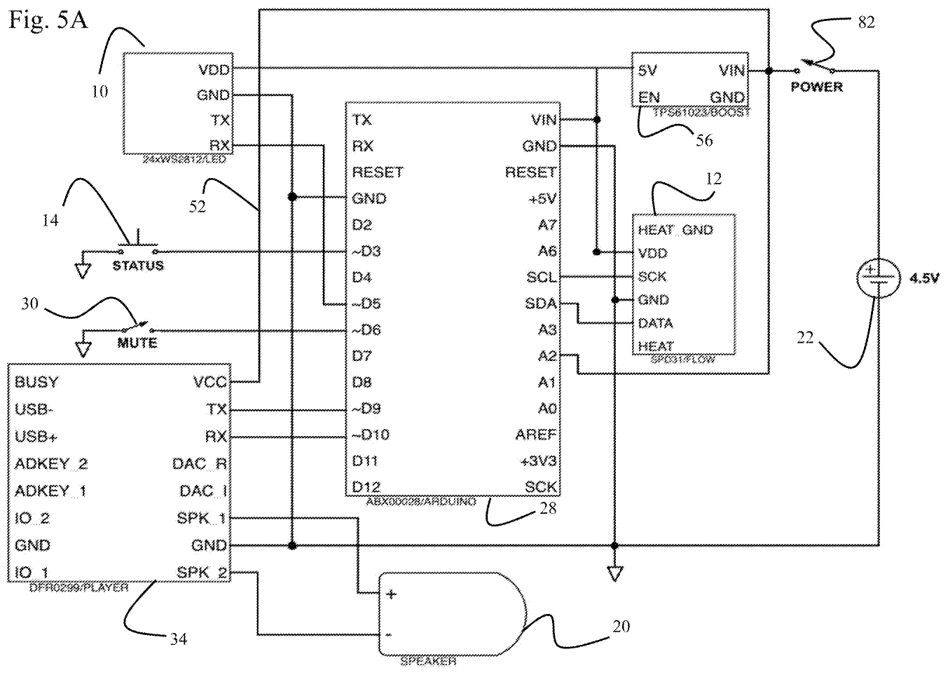

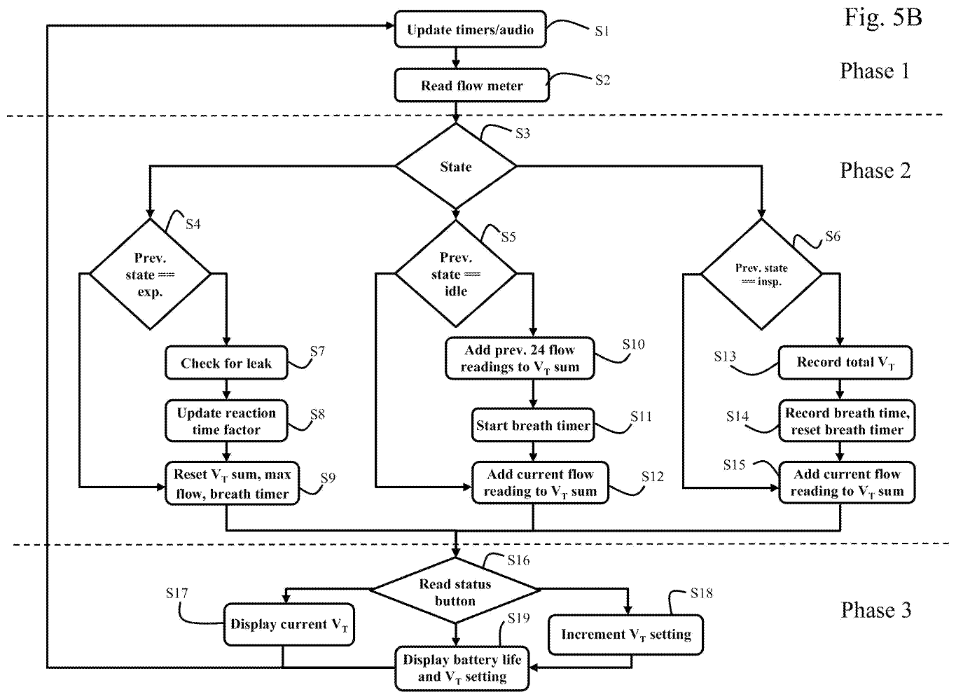

Respiratory tidal volume monitor and feedback device software. The following describes one embodiment of a method to electronically operate the respiratory tidal volume monitor and feedback device 2. After a setup routine to initialize serial and I2C channels, set I/O pin directions, and initialize hardware, the device's software operates on a continuous loop. The loop is separated into three main sections. First in step S1, timers and counters are updated based on results from the previous loop, and any cued audio is played. Data are then read from the mass flow sensor in step S2, which is used to determine the state of the respiratory tidal volume monitor and feedback device in step S3 (idle, inspiration, or expiration) and calculate tidal volumes delivered through the device in steps S4-S15. Finally, either the current delivered V_{T} or the device's status (target V_{T} setting and battery life) are displayed on the LED ring in step S19. The general structure of the software as described above is illustrated in FIG. 5B.

Audio Cues and Timers: In addition to displaying delivered tidal volumes, the respiratory tidal volume monitor and feedback device 2 preferably gives instruction and feedback on the rate and quality of respirations through audio cues from speakers In one embodiment, preferably every 6 seconds, for example, the respiratory tidal volume monitor and feedback device 2 plays an initiation audio cue saying, “Go,” or a similar initiation word, or a similar meaning word in the user's language if it is not English, which instructs the user to begin an inspiration. Playing this audio cue every 6 seconds results in a respiratory rate of 10 breaths per minute. If greater or less than breaths per minute is appropriate, the timing for the audio cues may be proportionally increased or decreased in frequency as appropriate. Halfway through the cycle, or at the 3-second mark in a 6-second cycle, the

respiratory tidal volume monitor and feedback device may play rate audio cues that instruct the user to “Bag faster” or “Bag slower” if the duration of inspiration falls outside of a duration of inspiration window, such as between 3.0 seconds and 0.2 seconds, more preferably between 2.0 seconds and 0.5 seconds. The duration of the previous inspiratory cycle is recorded using the value of the breath timer described above. If the previous inspiration lasted longer than 2 seconds, for example, the “Bag faster” rate audio cue may be played. If the previous inspiration lasted less than 0.5 seconds, for example, the “Bag slower” rate audio cue is played. Throughout inspiration, the device 2 also keeps track of the maximum flow value read through the mass flow sensor 12. The “Bag slower” cue may also play if the maximum flow value for the previous inspiration exceeded a maximum rate, such as 60 liters per minute, for example. The duration of respiration may be inversely proportional to a rate of respiratory air flow or respiratory rate.

Next, at approximately ⅔ the way through the cycle, or at the 4-second mark for a 6 second cycle for example, the respiratory tidal volume monitor and feedback device 2 may play a leak audio cue if an air leak is present in the system, such as “Leak detected”.

In one embodiment of the operation of the respiratory tidal volume monitor and feedback device 2 attached to a BVM system 54, the air inspiration path 70 travels from a bag inlet 72, through the bag 6, through a bag outlet 74 that connects to the flow channel tube 36, through the ventilation channel 44, into the mask 8 that is connected to the mask connector 42, and out of a patient engagement opening 76 of the mask 8. The air expiration path 78 is preferably identical but reversed to the air inspiration path for the first portion of the air expiration path 78. The air expiration path 78 begins at the patient engagement opening 76, moves through the mask 8, and into the ventilation channel 44 via the mask connector 42, and out of the respiratory tidal volume monitor and feedback device 2 through the flow channel tube 36, and into the bag outlet 74. At the bag outlet 74 though, the air expiration path 78 is routed through an exhalation port 80 and out of the BMV system 54. The respiratory tidal volume monitor and feedback device 2 records the total V_{T} for each inspiration and each expiration. Leaks are, as such a measurement may suggest that air is escaping the system after initially passing through the respiratory tidal volume monitor and feedback device 2. Such a leak or air escape may be if the mask 8 is not sealed firmly on the patient or CPR manikin 68.

Reading the Mass Flow Sensor and Determining State. In one embodiment of the disclosed invention, on each pass through the main software loop, steps S1-S19, the system processor reads one flow measurement from the mass flow sensor 12 over I2C 52 as raw data, then converts the data to standard liters per minute through a conversion formula provided by the SDP3x datasheet. The, for example, 25 most recent flow readings may be stored to aid in determining whether the device 2 is in an idle, inspiratory, or expiratory state. While in non-idle states, flow readings are used to calculate a running total of the delivered tidal volume. Volume can be calculated as the integral of flow rate over time, which may be approximated in software as a non-recursive trapezoidal Riemann sum approximation as follows:

V_{T}=½Σᵢ₌₁²⁵(fᵢ+fᵢ₋₁)(tᵢ−tᵢ₋₁) (1)

(where V_{T} is the tidal volume in mL and the f and t terms are the current and previous 25 flow and corresponding time measurements in mL/s and s, respectively) to begin the running total of delivered tidal volume. Throughout the rest of inspiration, a running total of tidal volume is calculated using a recursive trapezoidal Riemann sum approximation:

V_{T}_{ᵢ}=V_{T}_{_{i−1}}+½(fᵢ+fᵢ₋₁)(tᵢ−tᵢ₋₁)

(where the i^{th} and i−1^{st} terms are the current and previous measurements, respectively). When switching from inspiration to expiration and throughout expiration, identical recursive and non-recursive trapezoidal Riemann sum calculations are performed to calculate the expired volume.

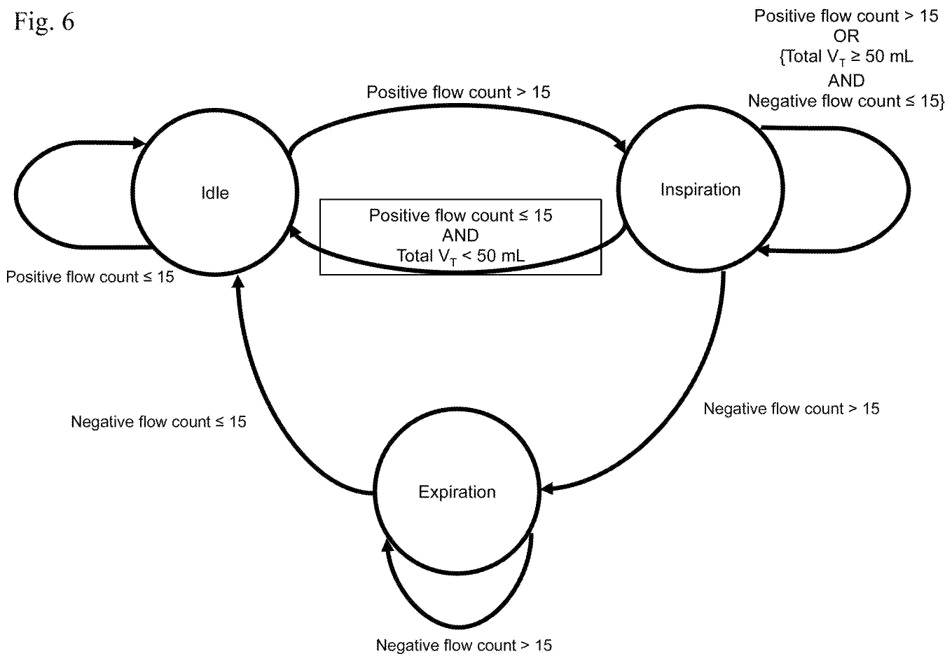

Turning to FIG. 6, the respiratory tidal volume monitor and feedback device 2 is a device that may function in 3 distinct states: (1) idle, (2) inspiration, and (3) expiration. One respiratory cycle may consist of moving consecutively from idle, to inspiration, to expiration, and finally back to idle state.

When first powered on, such as by depressing a power switch 82 for example, the system processor 24 begins the device 2 in the idle state. The device's state is reevaluated by the system processor 24 in every pass through the main software loop after reading data from the flow sensor 12. From idle, if a majority, such as more than 15 of the previous 25, flow readings are positive (air is flowing into the patient), the system processor 24 switches the device 2 to the inspiratory state. While in inspiratory, if a majority, such as more than 15 of the previous 25, flow readings are negative (air is flowing out of the patient), the system processor 24 switches the device 2 to the expiratory state. Finally, while in the expiratory phase if less than a majority or less than a super majority, such as 15 or fewer of the previous 25, flow readings are negative (air is no longer flowing out of the patient), the device switches from expiration to idle.

The mass flow sensor 12 is preferably particularly sensitive, such as 0.1 Pa for example. An effect of high sensitivity may be that small fluctuations in airflow through the device could lead to a “false positive” respiratory cycle progression (from idle, to inspiration, to expiration, and back to idle). To reduce this occurrence, an additional check at the inspiratory state is preferably added, where a low (such as less than 15 out of 25) positive flow count and a low V_{T} (such as less than 50 mL) leads to a reversion back to idle.

After updating the state, the system processor 24 performs a set of calculations based on the current and previous state. In the inspiratory and expiratory states, a running total of delivered tidal volumes is maintained in the system memory 26 by summing new flow measurements using the method described above. In the idle state, the running total for V_{T} is reset to 0. Upon switching from idle to inspiration, the system processor 24 initiates an inspiratory timer (to keep track of inspiratory time), and the previous 15 flow measurements are retroactively summed to begin a running total of delivered tidal volume. Upon switching from inspiration to expiration, the system processor 24 stops the inspiratory timer. If the inspiratory timer is less than 0.5 s, for example, a “Bag slower” audio cue is triggered. If the inspiratory timer exceeds 2 s, for example, the system processor may stop the inspiratory timer and immediately trigger a “Bag faster” audio cue. If the device reverts to the idle state from the inspiratory state, the inspiratory timer is reset to 0 and V_{T} is reset to 0. It is to be understood that statements in the disclosure of the device performing electronic actions shall be understood to be the system processor causing the device to perform such actions unless specified otherwise.

Furthermore, during expiration, a running total of exhaled volume is maintained by summing new flow measurements. When switching from expiration to idle, the system processor 24 compares expiratory volume to the inspiratory tidal volume. If the expiratory volume is less than 50% the inspiratory volume, a leak is detected. If between one and five, and preferably three consecutive leaks are detected, they system processor 24 triggers a “Leak detected” leak audio cue.

Additionally at this transition from expiration to idle, the system processor also preferably adjusts the displayed target tidal volume V_{T}_{_{display}} to compensate for the user's reaction time. Different users may react more quickly or slowly to the LED visual cues, and making slight adjustments to V_{T}_{_{display}} could account for this variability. In particular, a reaction time factor ratio r of the measured and target tidal volumes may be defined as:

$$r = \frac{{V}_{T}}{{V}_{{T}_{target}}}$$

(where V_{T} and V_{T}_{_{target}} are the measured and target tidal volumes, respectively). If r≥1.025, for example, the user has over-bagged. Consequently, in the next respiratory cycle, the displayed tidal volume V_{T}_{_{display}} may be adjusted slightly downward, by preferably between 5.0% and 0.5%, more preferably by 2% (r≥1.075) or 1% (1.025≤r<1.075), for example so that the user will stop inspiration sooner. The reverse is true when the user under-bags (r≤0.975): V_{T}_{_{display}} is adjusted upward by preferably between 5.0% and 0.5%, more preferably by 2% (r≤0.925) or 1% (0.975≤r<0.925) to encourage the user to deliver more air. These reaction-time adjustments to V_{T}_{_{display}} are preferably limited to changing above or below 20.0% of V_{T}_{_{target}}

Tidal Volume Settings, Battery Life, and Display: According to an embodiment, the final step of the main software loop is to update the device's LED ring 18 visual display 10. The respiratory tidal volume monitor and feedback device 2 may have two different display modes: the default tidal volume display (FIGS. 9A-9D) and a status display (FIG. 9E). The user may press the settings button 14 on the underside of the device 2 at any time, which will put the device 2 in status mode for a limited time period, such as 3 seconds. In the default display mode, the LED ring 18 displays the current delivered V_{T}. The user's V_{T} setting is adjusted based on the reaction time correction factor to yield the true target V_{T}. Each of the 24 pixels 62 in the LED ring 18 represents 1/24 of this target V_{T}. As a breath is delivered through the device, pixels 62 light up in a clockwise direction, with all 24 pixels 62 illuminated when the true target V_{T} has been reached. The pixels 62 glow green when 50% or fewer of the pixels 62 are illuminated, yellow when more than 50% but fewer than 100% of the pixels 62 are illuminated, and red when 100% of the pixels 62 are illuminated (when the target V_{T} has been reached). As air travels back through the device during expiration, the pixels 62 turn off in the counterclockwise direction in the LED ring 18 providing a ‘stoplight’ of respiratory cues. It is understood that a different number of pixels 64 may be provided in the LED ring 18, and the same inventive methods used, changing appropriately based on the different number of pixels.

In status mode, approximately half of the pixels 62 may be designated to show the device's 2 current V_{T}_{_{target}} while the other approximately half of the pixels 62 may display battery life. V_{T}_{_{target}} may be displayed through purple colored LED pixels 62 that align with the V_{T} of, for example, indicial numbers printed into the ring guard 60. Pushing the settings button 14 while status mode is still active may increment the V_{T}_{_{target}} setting by 50 mL, up to a maximum of 900 mL, for example. At the 900 mL setting, the next press may reset the V_{T} target setting to 300 mL, for example. Additionally, the system processor my determine the battery life by reading an analog input connected to the positive terminal of the batteries 22. The battery life may be determined to be “high,” “medium,” or “low,” with small drops in voltage indicating significant changes in battery capacity, as battery output voltages can remain high until the battery 22 is almost completely drained. In a low battery status, the system processor 24 may cause the LED ring 18 to display a segment of red pixels 62. In a medium battery status, the system processor may cause the LED ring 18 to display the segment of red pixels 62, plus a segment of yellow pixels 62. In a high battery status, the system processor 24 may cause the LED ring 18 to display red, yellow, and green segments. The respiratory tidal volume monitor and feedback device's 2 ring guard 60 may have battery life symbols that align with the LED pixel 62 segments for further ease of interpretation. Respiratory tidal volume monitor and feedback device Flow Measurement

Validation: An oxygen tank equipped with a two-stage pressure regulator was connected to the inlet sides of the flow sensor for the respiratory tidal volume monitor and feedback device and computer sensor arranged in series with silicone rubber fitted CPAP hosing (6 ft.×19 mm inner diameter, Philips Respironics, Murrysville, PA) and conventional corrugated ventilator tubing. The outlet side of the computer sensor was vented to atmosphere, allowing for unobstructed flow. The outlet pressure on the regulator was adjusted to achieve varying flow rates from approximately 0. to 90. standard liters per minute (slm) in 10. slm increments, as measured by the computer sensor. Standard liters per minute are defined for a gas at standard temperature and pressure (STP: 0° C., 100 kPa). The sensor assumes gases are at STP, which can cause slight discrepancies with true volume measurements at different temperatures and pressures; however, as both the computer and respiratory tidal volume monitor and feedback device use the same sensor, this error is not seen when comparing measurements from the two sources. The measured flow was recorded for 100 ms with both the respiratory tidal volume monitor and feedback device 2 (via serial communication with the computer) and with a standalone sensor run on the manufacturer's software, then the flow measurements were compared.

Respiratory tidal volume monitor and feedback device Flow Sampling Rate Validation. To determine the flow sampling rate achieved by the respiratory tidal volume monitor and feedback device 2, the respiratory tidal volume monitor and feedback device 2 was connected in-line between an adult BVM (SPUR II® adult model, AMBU® A/S, Columbia, MD, USA) and a calibrated test lung (Calibrated QuickLung®, IngMar Medical Pittsburg, PA, USA) with silicone rubber fitted CPAP hosing and conventional corrugated ventilator tubing. Breaths were then delivered to the test lung by manually squeezing the BVM bag 6, following the visual and audio cues presented to the user by the respiratory tidal volume monitor and feedback device 2 (target V_{T}=500 mL). The number of times that the main software loop was completed during 1-second intervals were recorded internally on the respiratory tidal volume monitor and feedback device 2 and sent to the computer, via USB transmission, during bagging. Since the flow is measured once per loop, the number of times the main software loop was traversed correlates one to one with the number of flow measurements made.

Simulation of Flow Sampling Rate Variation. To estimate the effect that altering sampling rate would have on tidal volume measurement accuracy, the computer flow sensor was connected in-line between the adult BVM and a test lung with silicone rubber fitted CPAP hosing and conventional corrugated ventilator tubing. A single breath was delivered via the BVM at a tidal volume of approximately 500 mL over an inspiratory time of approximately 1 second (measured inspiratory time 1.031 s), and the flow measurements (n=2151 samples) of the inspiratory portion of the respiratory cycle were recorded with the computer sensor. A flow waveform V(t) was then generated by piecewise linear interpolation of the flow measurements, and the tidal volume V_{T} was calculated by equation (1). Simulation of flow waveform generation for varying numbers of samples, n (where n varies from 3 to 10 samples in steps of 1, 10 to 100 samples in steps of 10, and 100 to 2100 samples in steps of 100) was achieved by dividing the inspiratory flow waveform \dot{V}(t) into n−1 equal intervals and calculating the flow at all interval boundaries to produce a new piecewise linear interpolation \dot{V}(t)ₙ. For each flow waveform \dot{V}(t)ₙ, the tidal volume V_{Tn} was calculated by equation (1), and the difference to V_{T} was calculated, and the calculation

ϵₙ=V_{T}−V_{T}_{ₙ}

was performed to determine he error ϵₙ between V_{T} and V_{T}_{ₙ}.

Validation of respiratory tidal volume monitor and feedback device Tidal Volume Measurement. To measure the respiratory tidal volume monitor and feedback device's 2 accuracy at measuring V_{T}, the respiratory tidal volume monitor and feedback device 2 and computer flow sensor were connected in-line in series between a mechanical ventilator (Engstrom Carestation™, General Electric Healthcare, Chicago, IL) and the test lung. The ventilator was set in mandatory volume control ventilation, and breaths were delivered to the test lung at a constant respiratory rate (10 breaths/min) and varying inspiratory times (tᵢₙₛₚ=0.5, 1.0, and 2.0 s) and tidal volumes (V_{T}=300 to 900 mL in 50 mL increments). Flow measurements from at least 10 consecutive respiratory cycles were captured with the computer flow sensor. The flow waveform was analyzed with custom code written in Python (version 3.7.2, Python Software Foundation, Beaverton, Oregon, United States) to determine inspiratory start and end times and calculate V_{T} via equation (1). The onboard, real-time calculation of V_{T} by the respiratory tidal volume monitor and feedback device 2 system processor 24 was sent to the computer at end inspiratory time via USB transmission. The computer and respiratory tidal volume monitor and feedback device 2 calculated V_{T}s were matched based upon end inspiration times, and the measurement differences in V_{T} were analyzed by Bland-Altman plot.

Respiratory tidal volume monitor and feedback device Audio Cue Validation. The audio cues “Bag slower” and “Bag faster” may be caused to be triggered by the system processor 24 when the measured tᵢₙₛₚ is too slow (e.g., tᵢₙₛₚ>2.0 s) or too fast (e.g., tᵢₙₛₚ<0.5 s), respectively. To validate that the audio cues are being triggered at an appropriately time and volume, the respiratory tidal volume monitor and feedback device 2 was again connected in-line to the test lung and mechanical ventilator. The ventilator was set in mandatory volume control ventilation (respiratory rate=5 breaths/min) at varying tidal volumes (V_{T}=300, 500, and 750 mL). To determine the tᵢₙₛₚ that the “Bag slower” audio cue was triggered for a given V_{T}, tᵢₙₛₚ was set to 0.5 s and allowed to cycle through at least 10 respiratory cycles. If the cue was triggered at least once out of the 10 respiratory cycles, then tᵢₙₛₚ was incrementally increased at the smallest increment achievable with the mechanical ventilator until the system processor 24 failed to trigger the cue. Then, tᵢₙₛₚ was incrementally decreased until the system processor 24 triggered the audio cue for all 10 respiratory cycles. This tᵢₙₛₚ was defined as the lower bound time for triggering the “Bag slower” audio cue. The upper bound time—the fastest tᵢₙₛₚ for which the “Bag faster” audio cue was not triggered in at least one respiratory cycle—was then the previous tᵢₙₛₚ increment. The average tᵢₙₛₚ that triggers the “Bag slower” cue lies between the lower and upper bound times. This process was repeated for the “Bag faster” audio cue with an initial tᵢₙₛₚ set to 2.0 s.

Respiratory tidal volume monitor and feedback device Leak Detection Validation. To test the sensitivity of the respiratory tidal volume monitor and feedback device's 2 leak detection algorithm, the ventilator was again set in mandatory volume control ventilation (respiratory rate=10 breaths/min, tᵢₙₛₚ=1.0 s) at varying tidal volumes (V_{T}=300, 500, and 750 mL). Holes of 1.5 mm diameter were sequentially drilled into the corrugated ventilator tubing between the expiratory side of the respiratory tidal volume monitor and feedback device's flow sensor and the test lung to cause increasing levels of leak, which was detected and calculated as a percent of V_{T} by the mechanical ventilator. If a leak was detected by the respiratory tidal volume monitor and feedback device 2, then a signal was transmitted to the computer from the respiratory tidal volume monitor and feedback device 2 via USB. The upper limit was defined as the percent leak for which the “Leak detected” signal was triggered 10 out of 10 respiratory cycles. The lower limit was then defined as the percent leak achieved at the previous number of drilled holes, where at least 1 out of the 10 respiratory cycles did not trigger a “Leak detected” leak audio cue.

Statistics. Data are presented as mean±standard deviation, unless otherwise noted. Pearson r correlation coefficients, log-log data transformation, simple linear regression analysis, and Bland-Altman analysis were performed with the built-in functions from Graphpad prism statistics software (version 9.3, Graphpad, San Diego, CA).

Results. The respiratory tidal volume monitor and feedback device 2 was successfully assembled and initialized. The respiratory tidal volume monitor and feedback device was successfully constructed from the 3D printed housing and electronic parts as designed. With the outer cover housing 4 removed, a mass flow sensor 12 could be installed into a flow sensor connector by hand and removed with relative ease. The 3D printed flow channel tube 36 could be tightly connected to the mass flow sensor, allowing for successful attachment of the respiratory tidal volume monitor and feedback device 2 between a BVM bag 6 and mask 8.

Flipping the “power” switch 82 successfully turned the respiratory tidal volume monitor and feedback device 2 on and the program initialized as expected, with the LEDs briefly lighting up blue to indicate powering on. However, during initial testing of the respiratory tidal volume monitor and feedback device, it became apparent that a triggered audio cue could not be delivered if all the LED pixels 62 were turned on at once, and the respiratory tidal volume monitor and feedback device 2 would subsequently power cycle. It became apparent that both the MP3 module 34 and LED ring 18 have substantial active current draw, and the current output of the 5V booster 56 with two AAA batteries 22 was not sufficient to support both components 34, 18. An additional AAA battery 22 was added to the battery bank to increase the voltage. However, due to space restriction, the use of an externalized battery pack was required in the prototype (not shown). After addition of a third AAA battery 22, the respiratory tidal volume monitor and feedback device 2 initialized as intended and did not shut off unexpectedly during subsequent tests.

The LED ring 18 produced signals appropriately during V_{T} delivery, subsequently lighting up and changing from green to yellow and finally to red (FIGS. 9A-9E). Depressing the “V_{T}/battery check” settings button 14 switched the device 2 to display mode, where the LED ring 18 lit up to display the current target V_{T}, as a partial ring of purple LED colored pixels 62 lit up to the current V_{T} label arranged around the periphery of the LED ring 18 on the ring guard, and current battery power, as an opposing partial ring of red/yellow/green LED colored pixels 62 (FIG. 9F). Additionally, the audio cues could be successfully muted and turned back on with the sequential depression of a mute switch audio control button 30. While in the embodiment shown, the audio control button 30 toggles audio on and off, in further embodiments it may cycle through off, low, medium, and high, and variation thereof.

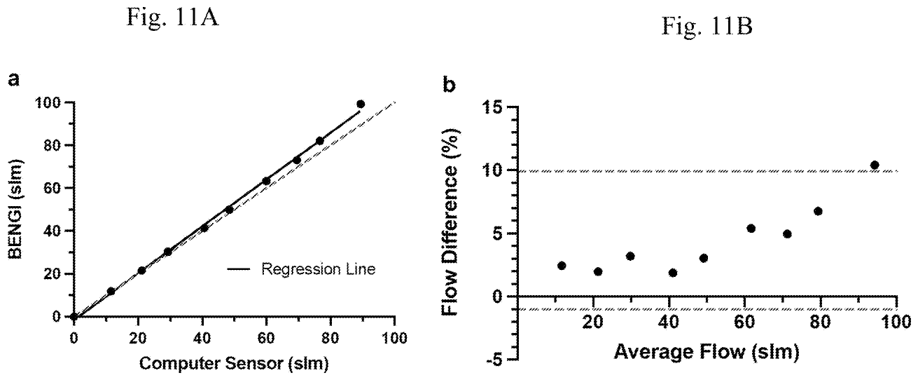

The respiratory tidal volume monitor and feedback device 2 accurately measures airflow rate. To determine the accuracy of flow rate measurements taken by the respiratory tidal volume monitor and feedback device 2, an oxygen tank with a pressure regulator was connected to the respiratory tidal volume monitor and feedback device 2 and computer sensor arranged in series. The pressure was adjusted to achieve varying flow rates, which was measured by both the respiratory tidal volume monitor and feedback device 2 and computer flow sensor (FIG. 11A). The measured flow readings demonstrated a high correlation between the respiratory tidal volume monitor and feedback device 2 and computer sensor (Pearson r>0.99). Bland-Altman analysis (FIG. 11B) showed a general trend towards an increase in percent difference between flow measurements with increasing flows and a tendency for the respiratory tidal volume monitor and feedback device 2 to overestimate, rather than underestimate, flow (95% limits of agreement from −1.0 to 9.9%). In particular, respiratory tidal volume monitor and feedback device 2 flow measurements were less than 5% different than the computer flow readings at values less than 60 slm, and the maximal difference in measured flow rate was 10.4% at 89 slm.

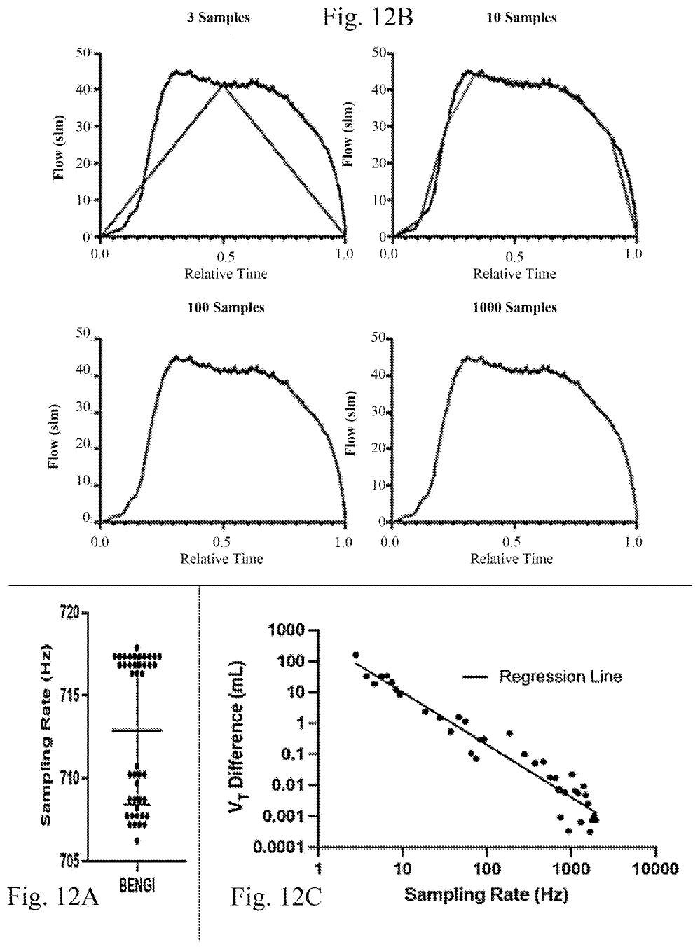

Flow sampling rate with the respiratory tidal volume monitor and feedback device 2 was sufficient for accurate tidal volume measurement. To determine the role that flow sampling rate played on the accuracy of the respiratory tidal volume monitor and feedback device's 2 V_{T} measurement, the respiratory tidal volume monitor and feedback device 2 was connected between a bag 6 and a test lung and subsequently bagged. The flow sampling rate of the respiratory tidal volume monitor and feedback device 2 during 1-second intervals (n=43) was measured, and the average flow sampling rate was 713±4 Hz (FIG. 12A). Interestingly, the sampling rate distribution appears bimodal.

In a subsequent test, a single breath was manually delivered via the BVM 54, and the flow was measured with the computer flow sensor (V_{T}=506 mL, tᵢₙₛₚ=1.031 s). Simulated flow waveforms f(t)ₙ were then generated from the measured flow waveform at varying simulation flow sampling rates n (FIG. 12B) and resultant V_{T}_{ₙ} and tidal volume difference to the maximum sampling rate (ϵₙ) were calculated. With increasing n, ϵₙ appeared to decrease linearly on a log-log scale (FIG. 12.C, Pearson r=−0.96). Extrapolation of this relationship was then used to predict the error of the respiratory tidal volume monitor and feedback device due to its lower flow sampling rate of 713 Hz, which was estimated to be 0.7 μL (FIG. 12C, red point).

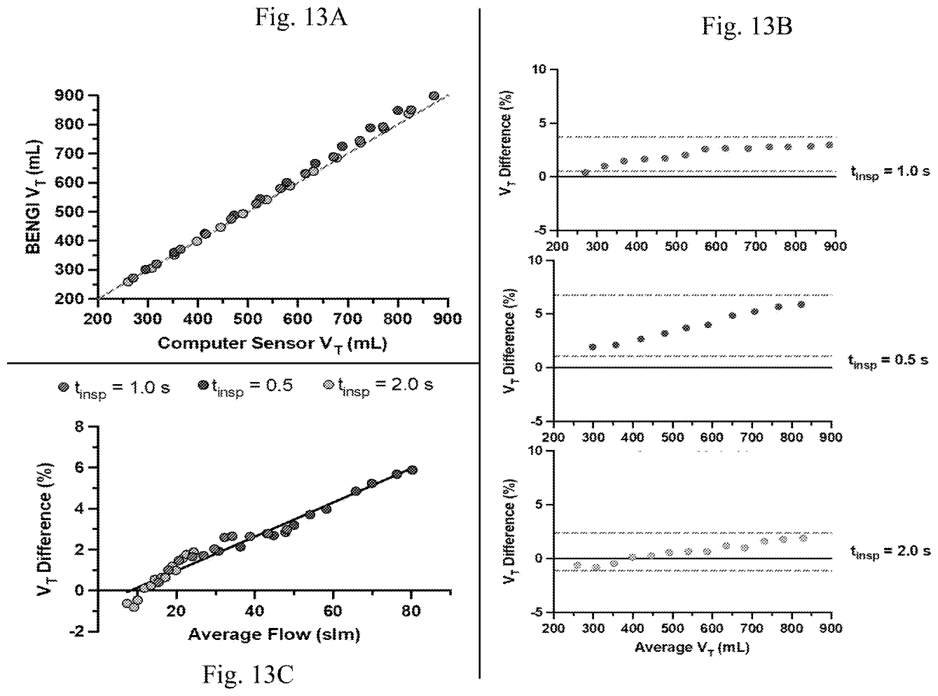

The respiratory tidal volume monitor and feedback device 2 accurately measured different tidal volumes delivered with a mechanical ventilator at varying inspiratory times. To test the accuracy of the respiratory tidal volume monitor and feedback device at measuring V_{T}, the respiratory tidal volume monitor and feedback device 2 and computer flow sensor was attached between the Carestation® mechanical ventilator and the test lung. The Carestation® was set for mandatory volume control ventilation at varying V_{T}s and inspiratory times. The respiratory tidal volume monitor and feedback device's 2 V_{T} calculations were recorded and compared to V_{T} computed from the flow waveform captured by the computer flow sensor (FIG. 13A). At tᵢₙₛₚ=0.5 s, set V_{T}s>750 mL could not be achieved by the mechanical ventilator because the maximum pressure allowed (100 cm H₂O) was exceeded. The calculated V_{T} from the respiratory tidal volume monitor and feedback device 2 were highly correlated (Pearson r>0.99) with the computed V_{T} from the computer sensor at all three inspiratory times (tᵢₙₛₚ=1.0, 0.5, and 2.0 s).

Bland-Altman analysis revealed that for all measured values, the V_{T} calculated by the respiratory tidal volume monitor and feedback device 2 differed from the V_{T} computed from the computer flow sensor by <7% (FIG. 13B; 95% limits of agreement=0.53 to 3.7%, 1.1 to 6.7%, and —1.1 to 2.3% for tᵢₙₛₚ=1.0 s, 0.5 s, and 2.0 s, respectively). Additionally, the respiratory tidal volume monitor and feedback device 2 overestimated V_{T} at the faster inspiratory times (tᵢₙₛₚ=1.0 and 0.5 s), and at the slower inspiratory time (tᵢₙₛₚ=2.0 s), the respiratory tidal volume monitor and feedback device 2 underestimated V_{T} at lower V_{T}s (<400 mL) and slightly overestimated at higher V_{T}s (>400 mL). When plotting the percent difference of V_{T} versus the average flow during each breath, a clear correlation (Pearson r=0.98) can be observed (FIG. 13C).

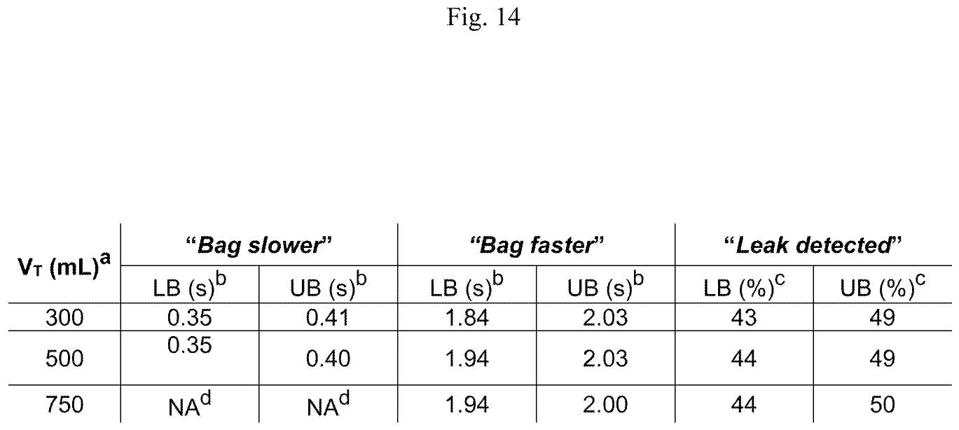

The respiratory tidal volume monitor and feedback device 2 audio cues properly triggered at varying tidal volumes. The upper and lower bounds for the tᵢₙₛₚ that triggered the “Bag faster” and “Bag slower” rate audio cues are shown in FIG. 14. The “Bag slower” rate audio cue was triggered at tᵢₙₛₚ slightly faster than the set trigger time value of 0.5 s at V_{T}=300 (between 0.35 to 0.41 s) and 500 mL (0.35 to 0.40 s). This means that tᵢₙₛₚ>0.5 s will not trigger the “Bag slower” rate audio cue at these V_{T}s. Additionally, there may be some tᵢₙₛₚ between the upper bound times and 0.5 s that will not trigger the “Bag slower” rate audio cue and remain undetected. The trigger for the “Bag slower” rate audio cue could not be tested at V_{T}=750 mL because the maximum pressure allowed by the mechanical ventilator was exceeded for tᵢₙₛₚ<0.5 s. The “Bag faster” rate audio cue was triggered at tᵢₙₛₚ between 1.84 to 2.03 s, 1.94 to 2.03 s, and 1.94 and 2.00 s for V_{T}=300, 500, and 750 mL, respectively. Thus, tᵢₙₛₚ greater than the upper bounds, which is ≤30 ms from the set trigger time of 2.0 s, will properly trigger the “Bag faster” rate audio cue at these V_{T}s. Furthermore, there may be some tᵢₙₛₚ between the lower bound times and 2.0 s that will inappropriately trigger the “Bag slower” rate audio cue.

The respiratory tidal volume monitor and feedback device 2 can accurately detect leaks at varying tidal volumes. The upper and lower bounds of percent leak that trigger the “Leak detected” signal at varying V_{T}s is also shown in FIG. 14 (between 43 to 49%, 44 to 49%, and 44 to 50% for V_{T}=300, 500, and 750 mL, respectively). Thus, for all leaks greater than the set leak value of 50%, the “Leak detected” signal is triggered at these V_{T}s.

Discussion, in this disclosure, the inventors disclosed and prototyped embodiments of the respiratory tidal volume monitor and feedback device 2, a hand held tidal volume feedback device preferably for use in manual ventilation scenarios. The respiratory tidal volume monitor and feedback device 2 was successfully assembled utilizing inexpensive, off-the-shelf electronic components and 3D printed parts for the housing. Two AAA batteries 22 and a 5V booster 56 were initially thought to provide adequate power for the respiratory tidal volume monitor and feedback device 2, yet initial testing demonstrated insufficient power when both the MP3 player 34 and LED ring 18 of the initial device 2 were powered simultaneously. For this reason, an additional AAA battery 22 was added, which lead to a design for an external battery pack 22 for the device 2. Future iterations of the respiratory tidal volume monitor and feedback device 2 design would include the possible addition of a rechargeable lithium-ion battery along with migration of electronic components to a single printed circuit board to allow for a more compact form factor, and even removing the need for the length provided by the mask connector 42 and thereby decreasing dead space.

Various tests were performed to validate the flow and tidal volume measurements along with appropriate audio cue triggering in the respiratory tidal volume monitor and feedback device 2 prototype. In particular, Bland-Altman analysis demonstrated that the prototype respiratory tidal volume monitor and feedback device 2 could measure tidal volumes within 7% of the computer sensor measurements in the wide range of tidal volumes and inspiratory times tested. Additionally, an increase in average measured flow was correlated with a rise in tidal volume discrepancy between the respiratory tidal volume monitor and feedback device 2 and computer-measured tidal volumes. We thought that this discrepancy could be due to either a difference in (1) flow measurements and/or (2) sampling rates between the computer and respiratory tidal volume monitor and feedback device 2 mass flow sensors 12. The measured sampling rate of the respiratory tidal volume monitor and feedback device 2 was 713 Hz, with all samples above 700 Hz. Our simple computational simulation of varying sampling rate predicted a log-log linear correlation between tidal volume difference and sampling rate, where an increase in sampling rate leads to a reduction in tidal volume difference. The tidal volume difference at the respiratory tidal volume monitor and feedback device's 2 sampling rate was predicted to be 0.7 μL, suggesting that sampling rate was not main cause for the observed differences in tidal volume measurements between the computer and respiratory tidal volume monitor and feedback device 2. However, when a constant flow was administered, there were marked differences between flow measurements with the respiratory tidal volume monitor and feedback device and computer sensor that increased with higher flow rates. This difference in measurements persisted even when the respiratory tidal volume monitor and feedback device 2 and computer flow sensors were switched (data not shown), indicating that the difference is not intrinsic to the calibration of the flow sensor themselves. Thus, the discrepancy in tidal volume measurements between the respiratory tidal volume monitor and feedback device 2 and computer is likely a flow-dependent, and not sampling rate-dependent, phenomenon.

The “Bag slower” audio was triggered by the system processor 24 at values slightly faster (˜100 ms faster) than the target 0.5 s at 500 mL and 300 mL tidal volumes. These bounds could not be tested at 750 mL since the flow rates required to achieve these fast inspiratory times in our lung model necessitated inspiratory pressures above mechanical ventilator's allowable pressure limit of 100 cm H₂O. The “Bag faster” audio cue was also triggered by the system processor 24 slightly faster (60 ms faster) than the target 2.0 s at 750 mL, within 60 ms faster or 30 ms slower at 500 mL, and within 160 ms faster or 30 ms slower at 300 mL. Further resolution to more precise upper and lower triggering time bounds could not be determined, as we were limited by the discrete inspiratory times that the mechanical ventilator provided for a given tidal volume. Additionally, the “Leak detected” signal was triggered by the systems processor somewhere between a 43 to 50% leak at all three tested tidal volumes, slightly lower than the target leak trigger of 50%.

Further determination of these upper and lower trigger time bounds could be achieved with a different external mechanical ventilator that allows for more precise inspiratory times and higher inspiratory pressures (for the “Bag slower” and “Bag faster” triggers) or by drilling with a smaller drill bit (for the “Leak detected” trigger). However, the upper and lower bounds demonstrated may arguably represent significant agreement to their respective targets, and any refinement likely do not represent a clinically relevant improvement.

In adult patients, rescue breaths during cardiopulmonary resuscitation may be administered either in tandem (asynchronously) or between pauses (synchronously) in chest compressions. When delivered asynchronously, manual ventilation is provided at a constant rate simultaneous with chest compressions without pause. The respiratory tidal volume monitor and feedback device's 2 audio cues are well-suited for providing asynchronous ventilation, as the recommended respiratory rate is 6 breaths/min, which would be provided when following the “GO” audio cue. In most cases, decreasing inspiratory times below 1 s increases the risk of delivering inspiratory pressures so high as to cause gastric regurgitation and aspiration. The rate audio cues help reduce the chance of this harm occurring. Additionally, delivering an inspiration over too long a period of time may leave an insufficient period of time for the lungs to adequately deflate during expiration, so the rate audio cues also helps diminish the likelihood of the dangers from overly long inspiration occurring. However, when used with synchronous ventilation, the respiratory tidal volume monitor and feedback device's 2 audio cues as described above, will in one embodiment preferably be muted, as every 6 second “Go” cues, for example, would need to be ignored, the user timing delivery of breaths for short pauses in chest compressions instead. Delivering rescue breaths over the recommended 1 s to 3 s time period in synchronous ventilation could potentially trigger the “Bag slower” cue if the inspiratory time is less than 0.5 s (a reasonable inspiratory time for a synchronous rescue breath that would result in an inspiratory to expiratory time ratio of 1:1), while the “Bag faster” cue likely would not be triggered at all, even if a faster bag compression is necessitated, as this cue only occurs at times greater than 2 s. However, with synchronous ventilation following the visual cues for proper tidal volume delivery, as described above, would still be beneficial. In a further embodiment, a “synchronous ventilation” mode is selectable, that will provide timed auditory prompting for both chest compression and ventilation during synchronous resuscitation at appropriate time intervals based on a selectable, programable, or preselected compressions to ventilations with a ratio (e.g., 30:2), in which the respiratory tidal volume monitor and feedback device 2 enters a fourth state of “compression” during compressions, and then loops back to an appropriately augmented synchronous active ventilation steps S4-S6.

The use of a simple ring of LED pixels 62 and spoken audio cues in the respiratory tidal volume monitor and feedback device 2 is believed to be unique. These cues may be more easily interpreted by the user versus outputting numerical respiratory parameters and textual commands on a display, especially when then medical personal using the device 2 is attending to an emergency and may be splitting her attention between operating the device 2 and monitoring and or delivering other care to the patient. Such simple audio and visual cues could reduce the mental workload on healthcare personnel during resuscitation scenarios—who are often simultaneously managing and performing other aspects of resuscitation protocols beyond manual ventilation—thereby improving the quality of delivered care. However, a clear drawback to such audio cues is whether they can be accurately heard during the resuscitation. The audio cues thus would preferably to be loud enough to be easily heard in a noisier environment, yet this comes with the tradeoff of becoming distracting. Thus, a further embodiment includes a volume control function for the audio cues.

Further embodiments of the are disclosed invention are as follows. More condensed electronic components and circuitry. Eliminate extra 3D-printed adapter. Reducing overall size improves respiratory tidal volume monitor and feedback device 2 ability to incorporate between the valve/bag outlet 74 and mask 8/advanced airway device. Use a rechargeable battery with recharging circuitry/ports as needed, instead of AAAs to reduce the need to remove housing between uses. Use inductive charging to eliminate external plugs for charging and possible plugging with dust, biologic debris, etc. Vapor and or liquid proof sealing electronic components within the device housing, allowing for the device 2 to be better cleaned/chemically sterilized between uses. Reduce the risk of incidental or intentional destruction/tampering of electronic components, with, for example, housing is made of tougher plastic, metal, or other material to reduce the risk of device damage from drops or rough use. Shock-proofing device 2 inside with addition of foam and or resin, or other shock proofing materials.

In further embodiments, there may be tracking and storing of performance metrics per user. The system processor may track variance and accuracy of delivered volumes and respiratory rates, and flow and volume waveforms to calculate more detailed metrics on flow rates, inspiratory times, etc. The device 2 can provide analytic feedback to a user's performance after a training session or resuscitation scenario, to continuously improve the user, or to help medical personnel diagnose the patient if the user had deviated from recommended bagging volume or rates.

In further embodiments, the device 2 may be equipped with wired or wireless communication capabilities, such as WiFi and/or Bluetooth transmitter and receiver, for example. This can aid in transmission of patient and or user data to external computers for use and storage. Further, a mobile computer app may be used to visualize performance data and battery levels, as well as choose settings for the device remotely (e.g., change tidal volume settings, rate settings, LED colors, mute, voice vs metronome only, adult vs neonate).

In further embodiments, an O₂ and/or a CO₂ sensor may be included in the ventilation channel 44 and electronically connected to the microcontroller unit 28 to allow measurement of tidal O₂ and/or a CO₂, to assess, in real time, the patient's condition and the physiological efficacy of ventilation. End tidal CO₂ measurements would provide real-time physiologic data on whether overall ventilation is appropriate or inappropriate for a given patient. End tidal CO₂ measurements could then be integrated into the algorithm to fine-tune target tidal volume and/or respiratory rate for a given patient. Further, a pressure valve and/or pressure sensor may be incorporated in the pressure monitoring to alert the user of high inspiratory, low expiratory pressures. This would also allow for the construction of pressure/volume loops and the measurements of “work” and “power” induced by the manual ventilation, other potentially important measurements that may be correlated to manual ventilation induced lung injury. If injury is detected, the device could send display an alert either auditorily, visually, or transmit such alert wirelessly to computer connected directly wirelessly or connected through the network wirelessly. Pressure, work, and power measurements could also be used to fine-tune target tidal volume and/or respiratory rate. This also allows for lung compliance to be measured, which can be of clinical importance. Inclusion of simultaneous end tidal CO₂ and pressure monitoring may allow fine-tune tidal volume and respiratory rate.

In further embodiments, an LCD or LE display may be used rather than or in addition to the LED ring 18 to transmit visual data. Such LCD or LE display could also be circular as the LED ring 18. Further embodiments many increase the number of LED pixels from 24 to a higher number, to provide greater resolution to achieved target tidal volumes, for example.

Further embodiments may use increased implementation of other audio cues, including, for example, an audio cue/alarm for the respiratory rate being too high or too low, an audio cue/alarm to indicate if end tidal CO₂ is too high or too low, an audio cue/alarm for too high a power or work measurement (if pressure is also measured), and an audio cue/alarm to indicate the measured compliance of the lungs (if pressure is also measured). The audio cues could also be displayed visually at the same time as the auditory cues are outputted, such as specified LEDs lighting up and text on a display being displayed, for example.

In further embodiments, the “reaction time adjustment” algorithm may use alternative and/or additional controller methods than using constant change in proportionality (1% or 2% increase/decrease, up to 20% change of base target tidal volume), such as a proportional-integral-derivative (PID) controller, for example.

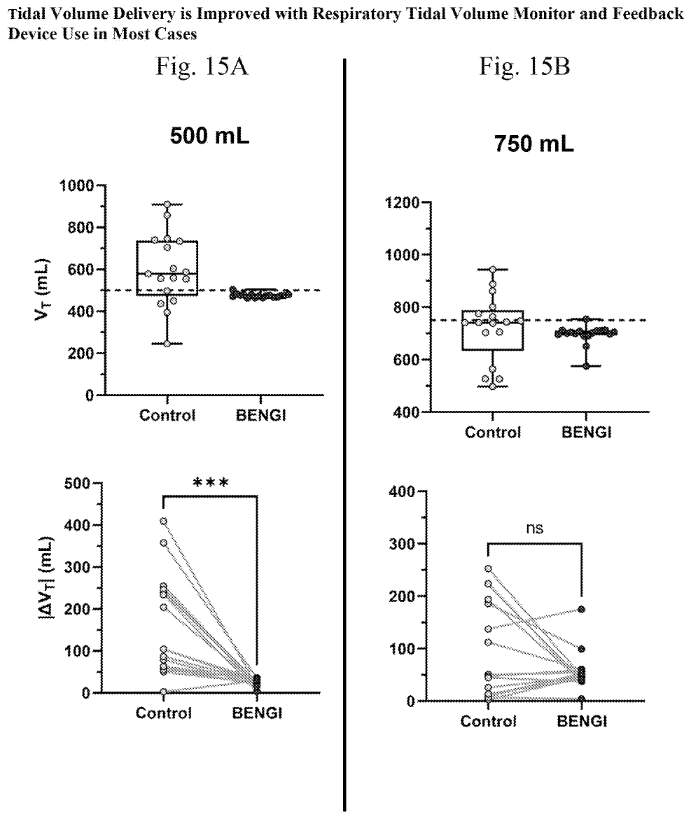

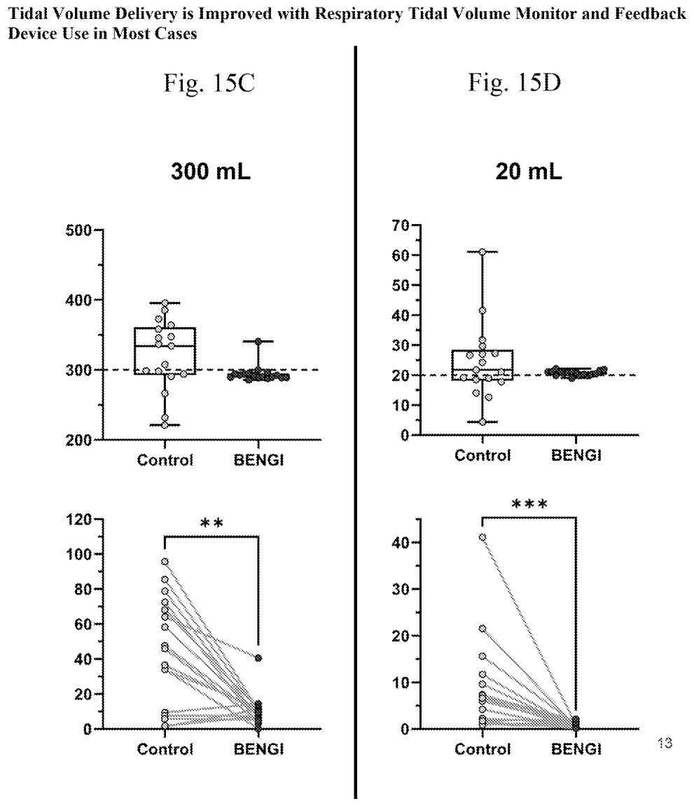

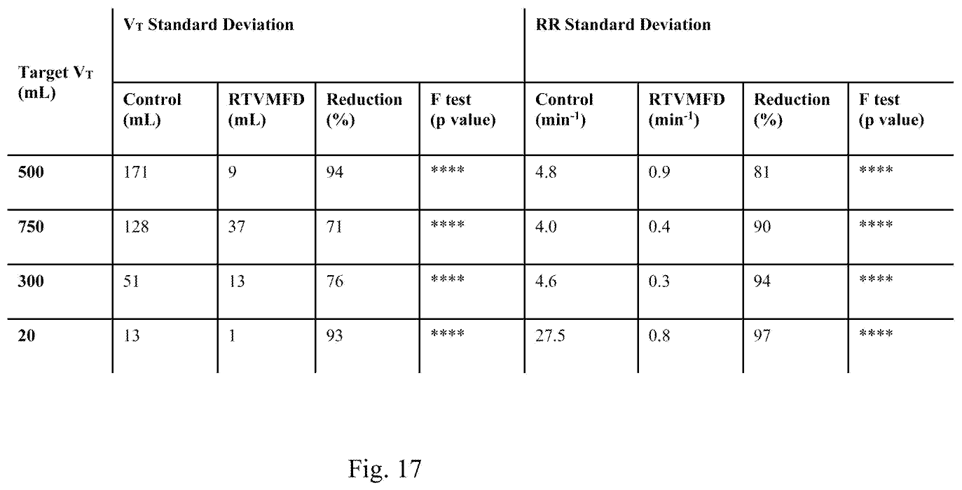

Turning now to FIGS. 15A-17, a training method is disclosed. Understanding that medical personnel may not always have a respiratory tidal volume monitor and feedback device 2 available when presented with a patient that needs artificial respiration, the inventors investigated the respiratory tidal volume monitor and feedback device 2 as a teaching tool to train medical personnel in repeatedly delivering exact levels of tidal volumes at set respiratory rates. This is based on developing fine motor muscle memory with real time feedback from preferably both auditory and non-textual visual cues. The inventors recruited 17 medical personnel to attempt to deliver different standard tidal volumes at set respiratory rates. As shown in FIG. 17, the reduction in standard deviation for results from control BMV to respiratory tidal volume monitor and feedback device 2 was shocking to the inventors: between 94% and 71% for different target tidal volumes delivered, and between 97% and 81% for set respiratory rates for the respective target tidal volumes. The results showed that use of the respiratory tidal volume monitor and feedback device 2 resulted in users almost uniformly improving accuracy delivering target standard tidal volumes at set respiratory rates, and sometimes dramatically so, evidencing that the respiratory tidal volume monitor and feedback device is an effective teaching tool.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

### Reference Numbers

2 Respiratory tidal volume monitor and feedback device (RTVMFD)

4 Housing

6 Bag

8 Mask

10 Visual display

12 Mass flow sensor

14 Settings button

16 Bottom of RTVMFD

18 LED Ring

20 Audio output

22 Power source

24 System processor

26 System memory

28 Microcontroller unit

30 Audio control button

32 Battery life circuit

34 Audio processor

35 Electronic connection for microcontroller unit

36 Flow channel tube

38 Frame

40 Clip

41 Fastener

42 Mask connector

44 Ventilation channel

46 Central axis

48 Pins

50 Pads

52 Bus

54 BVM system

56 Voltage booster

58 Top surface of frame

60 Ring guard

62 Pixel

64 Bottom surface of frame

66 V_{T} indicia

68 CPR manikin

70 Inspiration path

72 Bag inlet

74 Bag outlet

76 Patient engagement opening

78 Expiration path

80 Exhalation port

82 Power switch

Wherefore, I/we claim:

1. A respiratory tidal volume monitor and feedback device (RTVMFD) comprising:
a frame;
a flow channel tube attached to the frame;
a mass flow sensor disposed on the flow channel tube to detect air flow through the flow channel tube;
a microcontroller unit attached to the frame and electrically connected to the mass flow sensor via a bus, the microcontroller unit having a system processor, a system memory, and a system clock; and
a visual display attached to a top of the frame, the visual display being electrically connected to the bus.

2. The RTVMFD of claim 1 further comprising an audio output attached to the frame and electrically connected to the bus.

3. The RTVMFD of claim 2 wherein the visual display is a plurality of LED pixels.

4. The RTVMFD of claim 3, wherein the LED pixels are arranged in a ring.

5. The RTVMFD of claim 4, further comprising a plurality of target volume (V_{T}) indicia disposed on a ring guard, with each of the V_{T} indicia being adjacent to a respective LED pixel representing a respective target volume for the each of the V_{T} indicia.

6. The RTVMFD of claim 4 wherein the system processor is configured to execute instructions to:
receive mass flow data from the mass flow sensor during an inspiration;
cause the LED pixels to illuminate sequentially and in a proportional number to a tidal volume of inspirated air represented by the mass flow data.

7. The RTVMFD of claim 6 wherein the system processor is further configured to execute instructions to cause the LED pixels to change colors of illumination in response to the tidal volume of inspirated air.

8. The RTVMFD of claim 7 wherein the complete LED pixel ring represent 100% of a target tidal volume, and the LED pixels sequentially and proportionally illuminate forming an increasing arc length of illuminated LED pixels around a circumference of the ring as the measured tidal volume of inspirated air for each inspiration ranges from 0.0 mL to the target tidal volume.

9. The RTVMFD of claim 8 wherein the LED pixels change colors of illumination from a first color to a second color when the measured tidal volume reaches the target tidal volume.

10. The RTVMFD of claim 9 further comprising the system processor determining if the RTVMFD is in an expiration state, an idle state, or an inspiration state.

11. The RTVMFD of claim 10 further comprising when the RTVMFD is in an expiration state, the system processor checks for leaks by receiving expiration mass flow data from the mass flow sensor during a current expiration state, computing an expiratory tidal volume from the expiration mass flow data, and if the expiratory tidal volume is between 60.0% and 0.0% of an immediately previous inspiratory volume, determine that a leak is present.

12. The RTVMFD of claim 11 further comprising the system processor causing one of an audible cue, a visual cue, and both an audible cue and a visual cue to be generated when a leak is determined to be present.

13. The RTVMFD of claim 10 further comprising the system processor determining a flow rate of air during inspiration, and
when the flow rate is faster than an upper limit, determine that the inspiration rate is too fast, and
when the flow rate is slower than a lower limit, determine that the inspiration rate is too slow.

14. The RTVMFD of claim 13, wherein the system processor causes the speakers to
generate a verbal audio cue to alert the user to bag faster if the inspiration rate is determined to be too slow, and
generate a verbal audio cue to alert the user to bag slower if the inspiration rate is determined to be too fast.

15. A method of training a user to ventilate a patient comprising:
providing the user with a respiratory tidal volume monitor and feedback device (RTVMFD) and a Bag-Valve-Mask (BVM) functionally connected to one another, wherein the RTVMFD has
a frame;
a flow channel tube attached to the frame;
a mass flow sensor disposed on the flow channel tube to detect air flow through the flow channel tube;
a microcontroller unit attached to the frame and electrically connected to the mass flow sensor via a bus; and
a visual display attached to a top of the frame, with the visual display being electrically connected to the bus;
selecting a target tidal volume;
operating the BMV attempting to deliver the target tidal volume through the RTVMFD,
providing the user with substantially real time tidal volume feedback on success in reaching the target tidal volume with each inspiration delivered.

16. The method of claim 15 wherein the tidal volume feedback is in the form of an illumination of a progressive number of LED pixels arranged in a ring shape on a surface of the frame, where none of the LED pixels illuminated represents delivering a tidal volume of 0.0 mL and all of the LED pixels illuminated represents delivering a tidal volume equal to the target tidal volume.

17. The method of claim 16 wherein the tidal volume feedback is in the form of an illumination color change of the LED pixels when the target tidal volume is reached.

18. The method of claim 15 further comprising the system processor
tracking a duration of inspiration with each inspiration delivered;
determining if the duration of inspiration is less than, within, or greater than a target duration of inspiration window;
causing one of the visual display or a speaker to provide the user with substantially real time duration of inspiration feedback when the duration of inspiration is outside of the duration of inspiration window.

19. The method of claim 18 wherein the duration of inspiration feedback is
in the form of a verbal cue that the duration of inspiration is short when the duration of inspiration less that the duration of inspiration window; and
in the form of a verbal cue that the duration of inspiration is too long when the duration of inspiration is greater the inspiration window.

20. A respiratory tidal volume monitor and feedback device (RTVMFD) comprising:
a frame;
a flow channel tube attached to the frame, with a first end of the flow channel tube accessible for functional attachment to Bag-Valve-Mask (BVM) system bag outlet;
a mask connector, with a first end of the mask connector one of attached to and of unitary construction with a second end of the flow channel tube, and a second end of the mask connector accessible for functional connection to a BVM system mask;
a mass flow sensor disposed on the flow channel tube to detect air flow through the flow channel tube;
a microcontroller unit attached to the frame and electrically connected to the mass flow sensor via a bus, the microcontroller unit having a system processor, a system memory, and a system clock;
an audio processor electronically connected to the bus;
a visual display attached to a top of the frame, the visual display being electrically connected to the bus, wherein the visual display is plurality of LED pixels arranged in a ring shape;
a speaker attached to the frame and electrically connected to the bus;
a plurality of target volume (V_{T}) indicia disposed on a ring guard, with each of the V_{T} indicia being adjacent to a respective LED pixel representing a respective target volume for the each of the V_{T} indicia;
the system processor being configured to execute instructions to:
receive mass flow data from the mass flow sensor during an inspiration;
cause the LED pixels to illuminate sequentially and in a proportional number to a tidal volume of inspirated air represented by the mass flow data, wherein the complete LED pixel ring represents 100% of a target tidal volume, and the LED pixels sequentially and proportionally illuminate forming an increasing arc length of illuminated LED pixels around a circumference of the ring as the measured tidal volume of inspirated air for each inspiration ranges from 0.0 mL to the target tidal volume;
cause the LED pixels to change colors of illumination from a first color to a second color in response to tidal volume of inspirated air increasing from below 50.0% of target tidal volume to more than 50.0% of target tidal volume;
cause the LED pixels to change colors of illumination from the second color to a third color in response to tidal volume of inspirated air increasing from below 100.0% of target tidal volume to more than 100.0% of target tidal volume;
determine if the RTVMFD is in an expiration state, an idle state, or an inspiration state, and when the RTVMFD is in an expiration state, the system processor checking for leaks by receiving mass flow data from the mass flow sensor during an expiration, computing an expiratory tidal volume from the expiration mass flow data, and when the expiratory tidal volume is less than 50.0% of an immediately previous inspiratory volume, determine that a leak is present, and causing a verbal audible cue to be generated when a leak is determined to be present;
track a duration of inspiration with each inspiration delivered, determine if the duration of inspiration is less than, within, or greater than a target duration of inspiration window;
cause one of the visual display or speaker to provide the user with a substantially real time duration of inspiration feedback when the duration of inspiration is outside of the duration of inspiration window, wherein the duration of inspiration feedback is
in the form of a verbal cue that the duration of inspiration is short when the duration of inspiration less that the duration of inspiration window, and
in the form of a verbal cue that the duration of inspiration is too long when the duration of inspiration is greater the respiration rate window; and
cause the audio processor to cause the speaker to generate a verbal audio cue instructing the user bag initiate bagging at regular timed intervals.
